(19) United States
(12) Patent Application Publication
(10) Pub. No.: US 2026/0266837 A1
(43) Pub. Date: Sep. 10, 2026

(54) Methods for Cancer Recurrence Detection and Treatment Thereof

(71) Applicants: Institut Curie, Paris (FR); Centre National de la Recherche Scientifique, Paris (FR); Sorbonne Universite, Paris (FR)

(72) Inventors: Céline VALLOT, Paris (FR); Justine MARSOLIER, Brunoy (FR); Léa BAUDRE, Paris (FR); Pacôme PROMPSY, Montreuil (FR)

(73) Assignees: Institut Curie, Paris (FR); Centre National de la Recherche Scientifique, Paris (FR); Sorbonne Universite, Paris (FR)

(21) Appl. No.: 18/722,488

(22) PCT Filed: Jan. 3, 2023
(86) PCT No.: PCT/EP2023/050083

§ 371 Date: Jun. 20, 2024

(30) Foreign Application Priority Data
Jan. 3, 2022 (EP) 22305003.0

(51) Int. Cl. G01N 33/68; A61K 45/06; G01N 33/50

(52) U.S. Cl. CPC G01N 33/6875; A61K 45/06; G01N 33/5091; G01N 33/6893; G01N 2333/4727; G01N 2333/904; G01N 2800/54; G01N 2800/60; G01N 2800/7028

(57) ABSTRACT

The application concerns means for determining the risk of cancer recurrence in a human subject, in particular when the patient has or had therapy against cancer. In particular, the means of the invention involve determining the levels of expression of selected biomarkers, said selected biomarkers being selected among S100A2, LDHB, KRT14, TAGLN, NNMT, FOXQ1, FOSL1, NR2F2, KLF4 and TFCP2L1 in a sample previously obtained from the subject, and identifying the presence of persister cells within the sample when cancer cells express at least one of the selected biomarkers. The means of the invention also involve determining the presence or absence of persister cells, wherein persister cells has a quantity of H3K27me3 lower than a reference level. The invention also relates to method for treating patient who has or had cancer, and for preventing cancer recurrence in a patient who had or has cancer.

### TECHNICAL FIELD OF THE INVENTION

The application concerns means for determining the risk of cancer recurrence in a human subject, in particular when the patient has or had therapy against cancer. In particular, the means of the invention involve determining the levels of expression of selected biomarkers, said selected biomarkers being selected among S100A2, LDHB, KRT14, TAGLN, NNMT, FOXQ1, FOSL1, NR2F2, KLF4 and TFCP2L1 in a sample previously obtained from the subject, and identifying the presence of persister cells within the sample when cancer cells express at least one of the selected biomarkers. The invention also relates to method for treating patient who has or had cancer, and for preventing cancer recurrence in a patient who had or has cancer.

### BACKGROUND OF THE INVENTION

Residual disease, formed by cancer cells persistent to therapy, remains one of the major clinical challenges towards full cure^{1,2}. Undetectable residual cancer cells can persist in the body after treatment of a patient having a cancer and can eventually and unpredictably give rise to metastatic relapses. Some patients with metastatic cancers, who have positive response to treatment with cancer drugs (e.g. either partial or complete response), eventually relapse. These relapses can occur after months or even years even in the absence of detectable tumors following treatment. This is because of the existence of residual cells that are called persister cells or persistent cells, which create a reservoir that gives rise to what is called recurrent cancer. Chemotherapy or radiation may have killed most of the cancer cells, but some of them were either not affected or changed enough to survive the treatment and may become persister cells. A cancer that has recurred, usually after a period of time during which the cancer could not be detected, is called a recurrent cancer. The cancer may rise back to the same place as the original (primary) tumor or to another place in the body. Persister cancer cells are the discrete and usually undetected cells present within the initial tumors that survive cancer drug treatment and constitute a major cause of treatment failure. It has been proposed that persister cells can lead to the emergence of resistant clones through the acquisition of new mutations. However, the situation is more complex, as non-genetic mechanisms of resistance have been demonstrated in several cancer types. These observations imply that tolerant/persister cells can give rise to resistant clones through new specific mutations or by selecting a particular cell state that enables growth in the presence of the drug. Persister cells are usually characterized by their slow proliferation, adaptation to their microenvironment, and phenotypic plasticity. Mechanisms that underlie their persistence offer highly coveted and sought-after therapeutic targets, and include diverse epigenetic, transcriptional, and translational regulatory processes, as well as complex cell-cell interactions. The successful clinical targeting or detection of persistent cancer cells remains to be realized. Currently it is not possible to predict how likely a cancer is to recur. Determining if a cancer is likely to recur is a major concern since a recurring cancer may be harder to treat than the initial cancer, and/or may fast growing, and/or may widespread easily to other body parts. The major issue concerns the resistance to recurrent cancers to drug treatments. Most of the time, recurrent cancer has become resistant to treatment due to the persister cells that can grow and spread again. There are different types of cancer recurrence; local recurrence means that the cancer has come back in the same place it first started (for example, a breast cancer recurs in the breast); regional recurrence means that the cancer has come back in the lymph nodes near the place it first started; distant recurrence means the cancer has come back in another part of the body, some distance from where it started (often the lungs, liver, bone, or brain). A wide variety of mechanisms that contribute to the persistence of cancer cells have been reported. These mechanisms include epigenetic, transcriptional, and translational processes that are not mutually exclusive and may co-exist. According to the literature, there are several non-mutually exclusive strategies that are deployed by persistent cells to survive to treatment including slowing cell proliferation, adapting cell metabolism and changing cell identity.

To sum up, it is likely that cancer cells persist and become omnipresent after the initial clinical response of a patient to a treatment. Due to the existence of persister cells that evade the initial treatment, the patients are not fully cured but undergo a tumor-free period instead of a complete remission. This clinical reality highlights the urgent need to better understand the biology and mechanism of these persister cells with the aim of a) detecting them and b) destroying or neutralizing them. The clinician could use this determination to decide whether or not to administer treatment in order to treat those persister cells, and/or adapt the overall therapeutic strategy to avoid the risk of cancer recurrence.

In this context, being able to determine, in a reliable manner, the presence of persister cells in a given patient, and more particularly in a patient treated or under treatment against cancer is of crucial importance to the patient. In particular, there is a need to assess the presence of persister cells in breast cancers, and more particularly in triple-negative breast cancers, since persistence to chemotherapy results in the highest risk of recurrence among all breast cancer subtypes³.

### SUMMARY OF THE INVENTION

The invention relates to cancer recurrence determination, in particular to breast cancer recurrence determination, by identifying the presence of persister cells in a subject having cancer or who had cancer. The application pertains to means for detecting persister cells and/or for diagnosis of cancer recurrence. The inventors have identified genes the levels of expression of which are biomarkers of a type of cells that is associated with persister phenotype. More particularly, the inventors propose establishing the expression profile of at least one of these genes and using this profile as a signature of the persister phenotype within the cancer cells of the patient.

The means of the invention in particular use the determination by measurement or assay of the expression levels of at least one biomarker among a list comprising or consisting of selected biomarkers, in particular selected genes, the at least one of said biomarkers being selected among the list comprising or consisting of S100A2, LDHB, KRT14, TAGLN, NNMT, FOXQ1, FOSL1, NR2F2, KLF4 and TFCP2L1. Optionally, at least one other biomarker can be measured, in particular the level of methylation of H3K27, more particularly the amount of H3K27me3 associated with the promoter of the said biomarkers.

The invention thus relates to a method for determining a risk of cancer recurrence in a human subject who had or has a cancer, wherein the method comprises the steps of:
Determining in cancer cell, in particular tumor cells before or after exposure to a chemotherapeutic agent, more particularly tumor cells exposed to a chemotherapeutic agent, issued from a biological sample previously obtained from the subject whether at least one genetic biomarker selected from the group comprising or consisting of S100A2, LDHB, KRT14, TAGLN, NNMT, FOXQ1, FOSL1, NR2F2, KLF4 and TFCP2L1 is over-expressed as compared to a reference level,
Identifying the presence of persister cell in the biological sample as cancer cell over-expressing at least one genetic biomarker, in particular all genetic biomarkers, which expression has been measured,
Classifying the subject as being at risk to have cancer recurrence when persister cell has been identified in the biological sample.

The specific biomarkers listed herein have been well characterized and as illustrated in the results accompanying the present description, have been shown to predict relevant clinical outcomes of cancer persistence. Determining the expression of one or several of these biomarkers can thus be used as a clinical and diagnostic tool and may be useful in the determination of a suitable treatment for treating a patient having a cancer.

The means of the invention also relate to methods which comprise the determination by measurement or assay of the levels of expression of selected biomarkers and the subsequent treatment of the patient likely to have cancer recurrence.

### SHORT DESCRIPTION OF THE DRAWINGS

These and further aspects of the invention will be explained in greater detail by way of examples and with reference to the accompanying drawings in which:

FIG. 1. Identification of a pool of basal persister cells in tnbc in vivo and in vitro. a. Schematic representation of the standard of care for TNBC patients and the generation of patient-derived persister cells. b. Graph of the relative tumor volumes (RTV) over time (days). Colored growth curves correspond to tumors which have been further studied by scRNA-seq. Black arrows indicate the start of the second round of Capecitabine treatment for the corresponding mice. c. (Up) Phenotypes and cell numbers are indicated, with the number of mice used to collect samples in brackets. (Down) UMAP representation of PDX scRNA-seq datasets, colored according to sample of origin (first panel) or log2 gene expression signal for differentially expressed genes between persister cells and untreated tumor cells (remaining panels), log2FC and adjusted p-values are indicated above the graph. d. (Left) Venn diagram displaying the intersection of pathways activated in patient-derived persister cells from the 3 PDX models, among MSigDB c2_curated Breast/Mammary and c7_Hallmark pathways. P-value associated with the intersection is indicated below (exact test of multi-set intersections) (Right) Barplot displaying the top 5 pathways-for each category activated in patient-derived persister cells. x-axis corresponds to-log10 adjusted p-values for the model PDX_95. e. Graph representation of the cell proliferation of triple negative breast cancer cell line MDA-MB-468 (MM468) treated with 5-FU (green for persister cells, and orange lines for resistant cells) or with DMSO (untreated-grey lines). f. (Up) Schematic view of the experimental design. Experiment number and corresponding passage of cells at DO are indicated. (Down) UMAP representation of MDA-MB-468 cells scRNA-seq datasets, colored according to the sample of origin (first panel) or log2 gene expression signal for differentially expressed genes between persister cells (cluster R2) and untreated cells (cluster R10, KRT14 and TGFB1 panels) or for a differentially expressed gene between the two persisters clusters, i.e. clusters R4 vs R2 (CDH2 panel). Untreated population (in grey) corresponds to DMSO-D0- #1. g. (Left) UMAP representation of scRNA-seq as in 1f, restricted to cells with detected lineage barcode. Cells are colored according to lineage barcode and cluster membership is indicated (Extended FIG. 2c). R1, R2 correspond to RNA-inferred clusters. (Right) Scatter plot of the lineage barcode diversity detected in the scRNA-seq data across clusters and samples. Colors correspond to sample ID as in 1f. (Means are indicated for persister clusters R2 & R4. two-tailed Mann-Whitney test).

### FIG. 2. H3k27me3 Represses the Persister Expression Program Prior to

chemotherapy exposure. All experiments were performed in MDA-MB-468 cells. a. UMAP representation of scChIP-seq H3K27me3 datasets, cells are colored according to the sample of origin. Persister and resistant samples correspond to 5-FU-treated cells, days of treatment are indicated. b. Same as in a. with cells colored according to cluster membership. E1, E2 correspond to epigenomic-based clusters. c. Enrichment of H3K27me3 significantly depleted peaks in persister cells compared to all peaks across various gene annotation categories (see Methods). Full bars indicate adjusted p-value<1.0e-2. Empty bars indicate non-significant adjusted p-values. “PC” indicates protein coding genes. d. Repartition of H3K27me3 depleted peaks within log2 expression fold-changes quantiles from scRNA-seq experiments. e. Cumulative scH3K27me3 profiles over TGFB1 and FOXQ1 in untreated and persister cells (D33). Log2FC and adjusted p-value correspond to differential analysis of cells from cluster E1 versus cells from clusters E2+E4. f. Violin plot representation of the cell-to-cell inter-correlation scores between cells from clusters E1, E2 or E4 and cells from E1. Pearson's correlation scores were compared using a two-tailed Mann-Whitney test, p-value are indicated above plots. g. Dot plot representing log2 expression fold-change induced by 5-FU or EZH2i-1 at D33 versus D0. Pearson's correlation scores and associated p-value are indicated. h. Bulk H3K27me3 chromatin profiles for TGFB1 and FOXQ1 in cells treated with DMSO, 5-FU or EZH2i-1 at D33.

FIG. 3. Epigenomes of untreated cells are primed with co-accumulation of H3K27me3 and H3K4me3. a. UMAP representation of scChIP-seq H3K4me3 datasets, cells are colored according to the sample of origin-untreated cells (DO) and persister cells (D60). b. (Up) Cumulative scH3K4me3 enrichment profiles over FOXQ1 in untreated cells (DO) and persister cells (D60). ‘ns’ stands for not significant after differential testing comparing untreated and persister cells. (Down) H3K27me3->H3K4me3 and H3K27me3->IgG sequential ChIP-seq profiles of FOXQ1 in the untreated population. Comparative tracks show enrichment over IgG control with associated odd ratio and adjusted p-value. c. Doughnut chart displaying the fraction of persister genes (n=168) with H3K27me3 loss upon 5-FU treatment or with bivalent chromatin at TSS in untreated cells. Candidate master TFs—among persister genes—are indicated with the number of persister genes potentially regulated by the corresponding TF in parentheses. d. (Left) H3K27me3 and H3K4me3 chromatin profiles of human tumor samples for candidate master TF (Patient_39, Patient_95 and Patient_172). The percentage of tumoral cells are indicated for each sample. (Right) H3K4me3->H3K27me3 and H3K4me3->IgG sequential ChIP-seq profiles of untreated population of the three derived PDX_models (PDX_39, PDX_95 and PDX_172). Comparative tracks show enrichment over IgG control with associated odd ratio and adjusted p-value. e. H3K27me3 and H3K4me3 chromatin profiles for FOXQ1 of 6 additional human tumor samples. The percentage of tumoral cells are indicated for each sample. f. Dotplot showing the top pathways enriched in genes displaying a dual H3K27me3 and H3K4me3 enrichment in human tumor samples. Color of the dot corresponds to adjusted p-values and the size of the dot corresponds to the gene ratio, i.e. the fraction of bivalent genes belonging to this pathway. Stars indicate human tumor samples used to establish our PDX models

FIG. 4. EZH2 inhibition rescues cell fate biais upon chemotherapy exposure. All the experiments were performed in MDA-MB-468 cells. a. Histogram representing the number of cells after treatment with 5-FU alone or 5-FU and EZH2i over 21 days, relative to the number of cells at D0. Cells were pre-treated with EZH2i-1, inactive EZH2i-1 or EZH2i-2 for 10 days prior to chemotherapy treatment. (n=3, Mean±sd, Anova multiple comparisons test with Bonferroni's correction). b. Clustering of samples according to lineage barcode frequencies, detected by bulk analysis, using Spearman correlation score. c. UMAP representation of scRNA-seq datasets, colored according to the sample of origin. Cells were treated with DMSO (untreated) or with 5-FU alone (persister) or with 5-FU and EZH2i-1 (EZH2i-1 persister). d. UMAP representation of scRNA-seq datasets, barcoded cells were selected and colored according to hierarchical clusters e. (Left) UMAP representation of scRNA-seq datasets, barcoded cells were selected and colored according to lineage barcode. (Right) Histogram of the lineage barcodes diversity detected in the scRNA-seq data within hierarchical clusters, and across samples. Colors correspond to sample ID as in 4c. Ratio of unique barcodes/total barcodes and p-value are indicated (two-tailed Fisher test).

FIG. 5. Simultaneous KDM6i and chemotherapy treatment inhibits chemotolerance in vitro and delays recurrence in vivo. a. Colony forming assay at day 60 for 5-FU treated MDA-MB-468 cells in combination with DMSO or indicated concentrations of the KDM6i GSK-J4. b. Colony forming assay at day 60 for 5-FU treated MDA-MB-468 cells in combination or not with 1 μM of GSK-J4 or its inactive isomer GSK-J5, either simultaneously—added at D0—or added at day 39 of chemotherapy treatment. c. Relative tumor volumes for n=60 mice treated with either DMSO, GSK-J4, Capecitabine or a combination of Capecitabine and GSK-J4. Dashed line indicates the threshold to detect recurrent tumors, RTV=3. d. Kaplan-Meier plot of the overall disease-free survival probability since pathologic complete response pCR to initial treatment (tumor volume<20 mm3), number of mice treated and p-value are indicated (log-rank test).

### DETAILED DESCRIPTION OF SPECIFIC EMBODIMENTS OF THE INVENTION

In a first aspect, the invention relates to method for determining a risk of cancer recurrence in a human subject who had or has a cancer, wherein the method comprises the steps of:
Determining in cancer cell, in particular tumor cells before or after exposure to a chemotherapeutic agent, more particularly tumor cells exposed to a chemotherapeutic agent, issued from a biological sample previously obtained from the subject whether at least one genetic biomarker selected from the group comprising or consisting of S100A2, LDHB, KRT14, TAGLN, NNMT, FOXQ1, FOSL1, NR2F2, KLF4 and TFCP2L1 is over-expressed as compared to a reference level,
Identifying the presence of persister cell in the biological sample as cancer cell over-expressing at least one genetic biomarker, in particular all genetic biomarkers, which expression has been measured,
Classifying the subject as being at risk to have cancer recurrence when persister cell has been identified in the biological sample.

A cancer is a disease involving abnormal cell growth with the potential to invade or spread to other parts of the body. According to the invention, the cancer which affects or affected a patient may be selected from the list consisting of bladder cancer, bone cancer, brain cancer, breast cancer, cervical cancer, colon cancer esophageal cancer, gastric cancer, head & neck cancers, hodgkin's lymphoma leukemia, liver cancer, lung cancer, melanoma, mesothelioma, multiple myeloma myelodysplastic syndrome, non-hodgkin's lymphoma, ovarian cancer, pancreatic cancer, prostate cancer, rectal cancer, renal cancer, sarcoma, skin cancer, testicular cancer, thyroid cancer or uterine cancer. In a particular embodiment, the cancer which affect or affected a patient is a breast cancer, including breast cancer corresponding to ductal carcinoma, lobular carcinoma, invasive breast cancer, inflammatory breast cancer, metastatic breast cancer, hormone receptor positive breast cancer, hormone receptor negative cancer, HER2 positive breast cancer, HER2 negative breast cancer, triple-negative breast cancer. In a more particular embodiment of the invention, the cancer which affects or affected a patient is a triple-negative breast cancer.

Hormone receptor positive breast cancers express estrogen receptors (ER) and/or progesterone receptors (PR). Tumors that have estrogen receptors are called “ER positive.” Tumors that have progesterone receptors are called “PR positive.” Only 1 of these receptors needs to be positive for a cancer to be called hormone receptor positive. Cancers without these receptors are called “hormone receptor negative.

About 20% of breast cancers depend on the gene called human epidermal growth factor receptor 2 (HER2) to grow. These cancers are called “HER2 positive” and have many copies of the HER2 gene or high levels of the HER2 protein. HER2 positive cancers can also be either hormone receptor positive or hormone receptor negative. Cancers that have no or low levels of the HER2 protein and/or few copies of the HER2 gene are called “HER2 negative.” Triple-negative breast cancer (TNBC) is cancer that tests negative for estrogen receptors, progesterone receptors, and excess HER2 protein. Thus, triple-negative breast cancer does not respond to hormonal therapy medicines or medicines that target HER2 protein receptors. Still, other medicines need to be used to successfully treat triple-negative breast cancer. About 10-20% of breast cancers are triple-negative breast cancers. There is a growing interest in finding new medications that can treat this kind of breast cancer or interfere with the processes that cause TBNC to grow or to recure. Triple-negative breast cancer is considered to be more aggressive and has a poorer prognosis than other types of breast cancer, mainly because there are fewer targeted medicines that treat triple-negative breast cancer. It tends to be higher grade than other types of breast cancer. The higher the grade, the less the cancer cells resemble normal, healthy breast cells in their appearance and growth patterns. On a scale of 1 to 3, triple-negative breast cancer often is grade 3. A cancer recurrence corresponds to a clinical situation in a patient who has or has an initial cancer who is likely to redevelop a related cancer after complete or partial remission of the initial cancer. The patient may be or may be not treated for the initial cancer. According to the invention, the expression of at least one genetic biomarker is determined. The at least one genetic biomarker being selected from the group comprising or consisting of S100A2, LDHB, KRT14, TAGLN, NNMT, FOXQ1, FOSL1, NR2F2, KLF4 and TFCP2L1. In a particular embodiment, the expression of several genetic biomarkers selected from the group comprising or consisting of S100A2, LDHB, KRT14, TAGLN, NNMT, FOXQ1, FOSL1, NR2F2, KLF4 and TFCP2L1 are determined. In particular at least two, or at least three, or at least four, or at least five, or at least sic, or at least seven, or at least eight, or at least nine, or the ten genetic biomarkers selected from the group comprising or consisting of S100A2, LDHB, KRT14, TAGLN, NNMT, FOXQ1, FOSL1, NR2F2, KLF4 and TFCP2L1.

A biological marker (or biomarker) is defined as a biochemical, molecular, or cellular alteration that is measurable in biological media such as tissues, cells, or fluids, and that indicates normal or abnormal process of a condition or disease. The term “biomarker” refers to molecule which can be measured accurately and reproducibly, thereby leading to the provision of a “signature” that is objectively measured and evaluated as an indicator of normal biological processes, or pathogenic processes, or pharmacologic responses. In the context of the present invention, a biomarker corresponds to biological molecule(s) expressed by and/or present within cells of a human being. Thus, in the present invention biological markers include genetic biomarkers (corresponding to the transcript products of genes) and epigenetic biomarker (corresponding to methylation of DNA for example). In the present invention, biomarkers include DNA, RNA and proteins. The measure of the expression of the biomarkers leads to the provision of a signature that can be associated with the detection of cancer cells that are persister cells (i.e. cells that have resisted to a primary treatment against cancer and are likely to proliferate and spread later leading to cancer recurrence). In the context of the present invention, the term “persister cells” is used to describe cancer cells with non-mutational mechanisms of “resistance” to a cancer treatment, in particular chemotherapy. In the context of the present invention, a persistent cell is a cell that over-express at least one genetic biomarker among the list comprising or consisting of S100A2, LDHB, KRT14, TAGLN, NNMT, FOXQ1, FOSL1, NR2F2, KLF4 and TFCP2L1. Nevertheless, persister cancer cells can have the capacity to mutate to produce genetically altered, resistant clones later on.

The subject may be any human who had, has, develops, or is suspected to have or develop a cancer. In particular, the subject may be any human who had or has cancer, and has been or is treated with an anti-cancer therapy, like but not limited to chemotherapy, radiotherapy, immunotherapy, in particular chemotherapy. In a particular embodiment of the invention, the subject has or had a cancer selected among the breast cancers, in particular TNBC. In another particular embodiment, the subject has or had a breast cancer, and is or has been treated by chemotherapy, in particular has TNBC that is or has been treated by chemotherapy. The subject may be a child, an adolescent, an adult. The subject may or may not have been treated for symptoms associated with cancer. In a preferred embodiment of the invention, the subject is or has been treated against cancer, for example by chemotherapy, radiotherapy, immunotherapy or any suitable methods, in particular by chemotherapy, more particularly by thymidylate synthase (TS) inhibitor, more particularly fluorouacile (5-FU) or derivative thereof or analogue thereof. The method of the invention may optionally comprise determining one or more clinical factors of said subject, such as selected from sex, age, body mass index, health history.

A biological sample obtained from the patient can be any biological sample, such tissue, blood, urine, whole cell lysate. Methods of obtaining a biological sample are well known in the art and include obtaining samples from surgically excised tissue. Tissue, blood, urine and cellular samples can also be obtained without the need for invasive surgery, for example by puncturing the subject with a fine needle and withdrawing cellular material or by biopsy. In certain embodiments, samples taken from a patient can be treated or processed to obtain processed biological samples such as supernatant, whole cell lysate, or fractions or extract from cells obtained directly from the patient. In other embodiments, biological samples issued from a patient can also be used with no further treatment or processing. In a preferred embodiment, the biological sample obtained from the subject is a tissue, in particular a tissue from a tumor or a tumor extract, preferably obtained by biopsy. A biological sample issued from a subject may, for example, be a sample removed or collected or susceptible of being removed or collected from an internal organ or tissue or tumor of said subject, in particular from tumor, or a biological fluid from said subject such as the blood, serum, plasma or urine, in particular an intracorporal fluid such as blood. A biological sample collected or removed from the subject may, for example, be a sample comprising cancer cells which have been or are susceptible of being removed or collected from a tissue, in particular a tumor, of said subject. A step for lysis of the cells, in particular lysis of the cancer cells contained in said biological sample, may be carried out in advance in order to render nucleic acids or, if appropriate, proteins and/or polypeptides and/or peptides, directly accessible to the analysis.

In a particular embodiment, the biological sample is or is issued from a patient-derived xenograft (PDX). Cancer cells from the patient may be retrieved, and cultured through a graft in a receiving animal, in particular a mouse, for example according to the materiel and method disclosed herein. The PDX may be treated with chemotherapeutic agent, for example any chemotherapeutic agent disclosed herein, before performing the method according to the invention for assessing if persister cells are present within the PDX. PDX may be treated with a compound that reduces the quantity of H3K27me3 in cells in the PDX, like EZH2 inhibitor (EZH2i-1-UNC1999). Such a treatment may ease the determination of the presence of cells within the PDX which are likely to differentiate into the persister phenotype. Alternatively, the PDX may also be treated with any one of the following compounds before performing a method according to the invention:
a chemotherapeutic agent, radiotherapy or an immunotherapeutic agent, in particular a chemotherapeutic agent selected from the group comprising a thymidylate synthase (TS) inhibitor, more particularly fluorouacile (5-FU) or derivative thereof or analogue thereof; and/or
at least one inhibitor of S100A2, LDHB, KRT14, TAGLN, NNMT, FOXQ1, FOSL1, NR2F2, KLF4 or TFCP2L1; and/or
an inhibitor of lysine demethylase 6, more particularly of lysine demethylase 6 A/B or of H3K27me3 demethylase.

In a particular embodiment of the invention two PDXs may be issued from the patient, a first PDX being treated with any compound selected from the list consisting of:
a chemotherapeutic agent, radiotherapy or an immunotherapeutic agent, in particular a chemotherapeutic agent selected from the group comprising a thymidylate synthase (TS) inhibitor, more particularly fluorouacile (5-FU) or derivative thereof or analogue thereof; and/or
at least one inhibitor of S100A2, LDHB, KRT14, TAGLN, NNMT, FOXQ1, FOSL1, NR2F2, KLF4 or TFCP2L1; and/or
an inhibitor of lysine demethylase 6, more particularly of lysine demethylase 6 A/B or of H3K27me3 demethylase; and/or
a compound that reduces the quantity of H3K27me3 in cells, like EZH2 inhibitor,
   the second PDX being left untreated. The determination of the expression of at least one biological marker as listed herein may be compared between cells issued from the two PDXs, in particular to assess if persister cells are likely to differentiate from cancer cells in the PDX to persister cells following administration of one of the listed compounds.

The measurement or assay may be carried out in a biological sample which has been collected or removed from said subject and which has been transformed, for example by extraction and/or purification of proteins and/or polypeptides and/or peptides and/or DNA and/or RNA, or by extraction and/or purification of a protein fraction or cell fraction such as serum or plasma or cells extracted from blood.

Determining by measuring or assaying the level of expression of selected biomarkers may be carried out in a sample which has been obtained from said subject, such as a biological sample removed from or collected from said subject, or a sample comprising nucleic acids (in particular RNAs) and/or proteins and/or polypeptides and/or peptides of said biological sample, in particular a sample comprising nucleic acids and/or proteins and/or polypeptides and/or peptides which have been or are susceptible of having been extracted and/or purified from said biological sample, or

a sample comprising cDNAs which have been or are susceptible of having been obtained by reverse transcription of said RNAs.

One feature of the method according to the invention is that it includes the determination by measuring or assaying the level to which the selected biomarkers, in particular genes, are expressed in particular cells, in particular cancer cells, of said subject. The expression “level of expression of a gene” or equivalent expression as used here designates both the level to which this gene is transcribed into RNA, more particularly into mRNA, and also the level to which a protein encoded by that gene is expressed. The term “measure” or “assay” or equivalent term is to be construed as being in accordance with its general use in the field, and refers to quantification, in particular relative quantification. The level of transcription (RNA) of each of said biomarkers or the level of translation (protein) of each of said biomarkers, or indeed the level of transcription for certain of said selected biomarkers and the level of translation for the others of these selected biomarkers can be measured. In accordance with one embodiment of the invention, either the level of transcription or the level of translation of each of said selected biomarkers is measured. The fact of measuring (or assaying) the level of transcription of a biomarker includes the fact of quantifying the RNAs transcribed from that gene, more particularly of determining the concentration of RNA transcribed by that biomarker (for example the quantity of those RNAs with respect to the total quantity of RNA initially present in the sample. The fact of measuring (or assaying) the level of translation of a biomarker includes the fact of quantifying proteins encoded by that biomarker, more particularly of determining the concentration of proteins encoded by a gene corresponding to the selected biomarker(s), (for example the quantity of that protein per volume of biological fluid).

Certain proteins encoded by a mammalian gene, in particular a human gene, may occasionally be subjected to post-translation modifications such as, for example, cleavage into polypeptides and/or peptides. If appropriate, the fact of measuring (or assaying) the level of translation of a biomarker may then comprise the fact of quantifying or determining the concentration, not of the protein or proteins themselves, but of one or more post-translational forms of this or these proteins, such as, for example, polypeptides and/or peptides which are specific fragments of this or these proteins.

In order to measure or assay the level of expression of a biomarker, it is thus possible to quantify the RNA transcripts of a gene, or proteins expressed by a gene or post-translational forms of such proteins, such as polypeptides or peptides which are specific fragments of these proteins, or particular forms of proteins expressed, for examples epigenetic modifications including degree of methylation of one or several amino acid residues on proteins, in particular on DNA packaging protein Histone H3, more particularly tri-methylation of lysine 27 on histone H3 protein.

In order to measure the level of transcription of a biological marker, in particular a gene, its level of RNA transcription may be measured. Such a measurement may, for example, comprise assaying the concentration of transcribed RNA of each of said selected biological marker, either by assaying the concentration of these RNAs or by assaying the concentration of cDNAs obtained by reverse transcription of these RNAs. The measurement of nucleic acids is well known to the skilled person. As an example, the measurement of RNA or corresponding cDNAs may be carried out by amplifying nucleic acid, in particular by PCR. As an example, in the case of measurement of the level of expression of a gene by measurement of transcribed RNAs, i.e. in the case of measurement of the level of transcription of this gene, the measurement is generally carried out by amplification of the RNAs by reverse transcription and PCR (RT-PCR) and by measuring values for Ct (cycle threshold).

In order to measure the level of translation of a biological marker, in particular a gene, its level of protein translation may be measured. Such a measurement may, for example, comprise assaying the concentration of proteins translated from each of said selected genes (for example, measuring the proteins in cell extracts). Protein measurement is well known to the skilled person. As an example, the proteins (and/or polypeptides and/or peptides) may be measured by ELISA or any other immunometric method which is known to the skilled person, or by a method using mass spectrometry which is known to the skilled person. As an example, in the case of measuring the level of expression of a biological marker by measuring proteins expressed by the gene encoding the biological marker, i.e. in the case of measuring a level of translation of that gene, the measurement is generally carried out by an immunometric method using specific antibodies, and by expression of the measurements made thereby in quantities by weight or international units using a standard curve. Examples of specific antibodies are indicated in the examples of the present disclosure. A value for the measurement of the level of translation of a gene may, for example, be expressed as the quantity of this protein per volume of biological fluid, for example per volume of serum (in mg/mL or in μg/mL or in ng/ml or in pg/mL, for example) The measurement values are preferably values corresponding to the concentration or the quantity or the proportion of the levels of expression of each of said selected biological marker and reflect as accurately as possible, at least with respect to each other, the degree to which each of biological marker is expressed (degree of transcription or degree of translation), in particular by being proportional to these respective degrees. Thus, the expression level of biomarker in the biological sample may correspond to the proportion or the concentration or the quantity of the biomarker. The proportion may be expressed as a percentage (%) of the biomarker with the overall amount of protein within the sample or in respect to the other biological marker which expression is measured. Alternatively, instead of determining a proportion, a concentration or a quantity of the biological marker within the sample may be measured. The determination of the over-expression of at least one marker among the list of selected biomarkers is made by comparison with a reference level. The determination of over-expression of a biomarker in said subject may be deduced or determined by comparing the determined value(s) of each measured biomarker obtained from said subject with the value(s) associated with the same biomarker, or the distribution of the value(s) associated with the same biomarker, in reference subject(s) (for example a healthy subject, or healthy cells of the subject from whom the biological sample is issued, in particular healthy cells issued from the organ of the subject from whom the biological sample is issued), or cohorts of subjects which have already been set up as their likeliness to have cancer recurrence, in order to classify the subject into that of those reference cohorts to which it has the highest probability of belonging (i.e. to determining if the subject is likely to have cancer recurrence or not). Alternatively, the determination of over-expression of a biomarker in said subject may be deduced or determined by comparing the determined value(s) of each measured biomarker obtained from said subject with the value(s) associated with the same biomarker in reference cells known not to be persister cells as defined herein, like healthy cells or cancer cells which are sensitive to therapeutic treatment against cancer. The determination of the expression of the selected biomarker made on the subject and on the reference may correspond to measurements of the levels of gene expression (transcription or translation).

Overexpression of a biomarker may correspond to excessive expression (transcription or translation) of a biomarker of at least 10% as compared to the reference level, in particular at least 20% as compared to the reference level, in particular in particular at least 30% as compared to the reference level, in particular at least 40% as compared to the reference level, and more particularly at least 50% as compared to the reference level.

A reference level of a particular form of protein, for example epigenetic modified protein, in particular H3K27me3, a particular form of the DNA packaging protein Histone H3 indicating the tri-methylation of lysine 27 on histone H3 protein, in particular the quantification of H3K27me3 associated with the promoters of genes encoding the biological markers listed in the present description, may correspond to the quantity of the particular form of the considered protein in a reference cell, like healthy cells or cancer cells which are sensitive to therapeutic treatment against cancer.

Over expression of a biological marker is relative to a reference level. Thus, if according to the reference level, a marker is not expressed (for example when a reference marker is not expressed by healthy cells or by cells which are not likely to differentiate into the persister phenotype), over expression of a biological marker may correspond to the expression of the biological marker.

Biomarker S100A2 may correspond to the human gene S100A2 which may correspond to NCBI Entrez Gene reference No. 6273. Gene S100A 2 encodes protein S100 Calcium Binding Protein A2 (referenced S100-A2 or S100A2) and may correspond to Uniprot reference P29034.

Biomarker LDHB may correspond to the human gene LDHB (for Lactate DeHydrogenase B) which may correspond to NCBI Entrez Gene No. 3945. Gene LDHB encodes the protein L-lactate dehydrogenase B chain which may correspond to Uniprot reference P07195.

Biomarker KRT14 may correspond to the human gene KRT14 which may correspond to NCBI Entrez Gene No. 3861. Gene KRT14 encodes the protein Keratin 14 (KRT14) which may correspond to Uniprot reference P02533.

Biomarker TAGLN may correspond to the human gene TAGLN which may correspond to NCBI Entrez Gene No. 6876. Gene TAGLN encodes the protein Transgelin (TAGLN) which may correspond to Uniprot reference Q01995.

Biomarker NNMT may correspond to the human gene NNMT which may correspond to NCBI Entrez Gene No. 4837. Gene NNMT encodes the protein encoding Nicotinamide N-Methyltransferase (NNMT) which may correspond to Uniprot reference P40261.

Biomarker FOXQ1 may correspond to the human gene FOXQ1 which may correspond to NCBI Entrez Gene No. 94234. Gene FOXQ1 encodes the protein Forkhead Box Q1, a transcription factor, which may correspond to Uniprot reference Q9C009.

Biomarker NR2F2 may correspond to the human gene NR2F2 which may correspond to NCBI Entrez Gene No. 7026. Gene NR2F2 encodes the protein Nuclear Receptor Subfamily 2 Group F Member 2 (NR2F2), a transcription factor, which may correspond to Uniprot reference P24468.

Biomarker KLF4 may correspond to the human gene KLF4 which may correspond to NCBI Entrez Gene No. 9314. Gene KLF4 encodes the protein Kruppel Like Factor 4 (KLF4) which may correspond to Uniprot reference 043474.

Biomarker TFCP2L1 may correspond to the human gene TFCP2L1 which may correspond to NCBI Entrez Gene No. 29842. Gene TFCP2L1 encodes the protein Transcription Factor CP2 Like 1 (TFCP2L1) which may correspond to Uniprot reference Q9NZI6.

Biomarker FOSL1 may correspond to the human gene FOSL1 which may correspond to NCBI Entrez Gene No. 8061. Gene FOSL1 encodes the protein Fos-related antigen 1 (FRA1), which may correspond to Uniprot reference P15407.

Biomarker H3K27me3 may correspond to an epigenetic modification to the DNA packaging protein Histone H3 indicating the tri-methylation of lysine 27 on histone H3 protein. Human histone H3 may be encoded by the human gene H3-3A which may correspond to NCBI Entrez Gene No. 3020. Gene H3-3A encodes the protein Histone 3 A which may correspond to Uniprot reference P84243. In a particular embodiment, only H3K27me3 associated with the promoters of genes encoding the biological markers listed in the present description is quantified.

In accordance with the invention, persister cells are identified when cells, in particular cancer cells, more particularly cancer cells which have been treated or not with a chemotherapeutic agent or by radiotherapy or with an immunotherapy agent, and still more particularly cancer cells exposed to a chemotherapeutic agent, issued from the biological sample as cells over-expressing at least one biological marker selected from the list comprising or consisting of S100A2, LDHB, KRT14, TAGLN, NNMT, FOXQ1, FOSL1, NR2F2, KLF4 and TFCP2L1.

In a particular embodiment of the invention, persister cells are detected within the biological sample as cells over-expressing at least two, or at least three, or at least four, or at least five, or at least six, or at least seven, or at least eight, or at least nine, or the ten biological markers selected from the list comprising or consisting of S100A2, LDHB, KRT14, TAGLN, NNMT, FOXQ1, FOSL1, NR2F2, KLF4 and TFCP2L1. It should be noted that the method of the invention does not require determining the expression of all biological markers selected from the list comprising or consisting of S100A2, LDHB, KRT14, TAGLN, NNMT, FOXQ1, FOSL1, NR2F2, KLF4 and TFCP2L1 to assess if persister cells are present within the sample. The overexpression of a single biological marker may be sufficient to assess if persister cells are present within the biological sample. In a particular embodiment of the invention, the method comprises a step of determination of the expression of at least two, or at least three, or at least four, or at least five, or at least six, or at least seven, or at least eight, or at least nine, or all biological markers selected from the list comprising or consisting of S100A2, LDHB, KRT14, TAGLN, NNMT, FOXQ1, FOSL1, NR2F2, KLF4 and TFCP2L1. According to this embodiment, persister cells are identified as cells overexpressing at least one of the listed biological markers. In a preferred embodiment, the persister cells are identified as cells overexpressing at least two of the listed biological markers, or at least three of the listed biological markers, or at least four of the listed biological markers, or at least five of the listed biological markers, or at least six of the listed biological markers, or at least seven of the listed biological markers, or at least eight of the listed biological markers, or at least nine of the listed biological markers, or the ten listed biological markers.

Persister cells may also have the following properties: persister cells may survive initial chemotherapy treatment, and/or may be cells in the G0/G1 stage of their cellular cycle, and/or may be non-dividing cells (with an infinite doubling time).

In a particular embodiment of the invention, persister cells are identified as cells over expressing at least S100A2 and LDHB.

In a particular embodiment of the invention, persister cells are identified as cells over expressing at least KRT14, TAGLN, and NNMT.

In a particular embodiment of the invention, persister cells are identified as cells over expressing at least FOSL1 and KLF4.

In a particular embodiment of the invention, persister cells are identified as cells over expressing at least FOXQ1, FOSL1, NR2F2, KLF4.

In a particular embodiment of the invention, persister cells are identified as cells over expressing at least FOXQ1, FOSL1, NR2F2, KLF4 and TFCP2L1.

In a particular embodiment of the invention, persister cells are identified as cells over expressing:
at least S100A2 or LDHB, and
at least KRT14 and/or TAGLN and/or NNMT.

In a particular embodiment of the invention, persister cells are identified as cells over expressing:
at least S100A2 or LDHB, and
at least FOXQ1 and/or FOSL1 and/or NR2F2 and/or KLF4 and/or TFCP2L1.

In a particular embodiment of the invention, persister cells are identified as cells over expressing:
at least KRT14 and/or TAGLN and/or NNMT, and
at least FOXQ1 and/or FOSL1 and/or NR2F2 and/or KLF4 and/or TFCP2L1.

In a particular embodiment of the invention, persister cells are identified as cells over expressing:
at least S100A2 or LDHB, and
at least KRT14 and/or TAGLN and/or NNMT, and
at least FOXQ1 and/or FOSL1 and/or NR2F2 and/or KLF4 and/or TFCP2L1.

In a particular embodiment of the invention, persister cells are identified as cells over expressing:
at least S100A2 and LDHB; and/or
at least KRT14, TAGLN, and NNMT; and/or
at least FOSL1 and KLF4, in particular at least FOXQ1, FOSL1, NR2F2 and KLF4, and more particularly at least FOXQ1, FOSL1, NR2F2, KLF4 and TFCP2L1.

In a particular embodiment of the invention, persister cells are identified as cells over expressing:
at least one biological marker selected from the group comprising or consisting of least S100A2 and LDHB; and
at least one biological marker selected from the group comprising or consisting of at least KRT14, TAGLN, and NNMT;
at least one biological marker selected from the group comprising or consisting of at least FOSL1 and KLF4, in particular at least FOXQ1, FOSL1, NR2F2 and KLF4, and more particularly at least FOXQ1, FOSL1, NR2F2, KLF4 and TFCP2L1.

In a particular embodiment of the invention, the method further comprises the quantification of H3K27me3 within cancer cell. Persister cells are cells which have a quantity of H3K27me3 lower than a reference level.

In a particular embodiment of the invention, the method comprises the determination of the expression of at least one, at least two, or at least three, or at least four, or at least five, or at least six, or at least seven, or at least eight, or at least nine, or all biological markers selected from the list comprising or consisting of S100A2, LDHB, KRT14, TAGLN, NNMT, FOXQ1, FOSL1, NR2F2, KLF4 and TFCP2L1, and the quantification of H3K27me3 in cells issued from the biological sample, a persister cell being identified as cells expressing at least one biological marker as compared to a first reference level, and having a quantity of H3K27me3 lower than a second reference level.

In a particular embodiment of the invention, persister cells are identified as cells over expressing at least S100A2 and/or LDHB, and having a quantity of H3K27me3 lower than a reference level.

In a particular embodiment of the invention, persister cells are identified as cells over expressing at least KRT14 and/or TAGLN and/or NNMT, and having a quantity of H3K27me3 lower than a reference level.

In a particular embodiment of the invention, persister cells are identified as cells over expressing at least FOXQ1 and/or FOSL1 and/or NR2F2 and/or KLF4 and/or TFCP2L1, in particular FOSL1 and KLF4, in particular at least FOXQ1, FOSL1, NR2F2 and KLF4, and more particularly at least FOXQ1, FOSL1, NR2F2, KLF4 and TFCP2L1 and having a quantity of H3K27me3 lower than a reference level.

In a preferred embodiment of the invention, persister cells are identified when they over express as compared to a reference level the following biological markers: KRT14, TAGLN, NNMT, FOXQ1, FOSL1, NR2F2, KLF4 and TFCP2L1. This embodiment is in particular suitable to determine after treatment of a primary cancer if cells of the tumor are likely to become persister.

In a preferred embodiment of the invention, persister cells correspond to cells present within a tumor of the primary cancer in a patient, said persister cells over expressing the two biological biomarkers S100A2 and LDHB as compared to a reference level. This embodiment is in particular suitable for determining in primary cancer if this cancer is likely to become recurrent after treatment of the cancer.

In a particular aspect of the invention, the method is performed in vitro or ex vivo.

In another embodiment of the invention, it is provided a method for determining a risk of cancer recurrence in a human subject who had or has a cancer, wherein the method comprises the steps of:
Determining in cancer cell, in particular tumor cells exposed to a chemotherapeutic agent, issued from a biological sample previously obtained from the subject the presence or the absence of persister cells, wherein a persister cell has a quantity of H3K27me3 lower than a reference level,
Classifying the subject as being at risk to have cancer recurrence when persister cells have been identified in the biological sample.

The quantity of H3K27me3 may be assessed according to the present disclosure, and the reference level corresponds to any definition of the H3K27me3 reference level defined herein.

In another embodiment of the invention, it is provided a method for determining a risk of cancer recurrence in a human subject who had or has a cancer, wherein the method comprises the steps of:
Identifying cancer cells, in particular tumor cells exposed to a chemotherapeutic agent, issued from a biological sample previously obtained from the subject, that over-express at least one genetic biomarker selected from the group comprising or consisting of LDHB, S100A2, KRT14, TAGLN, NNMT, FOXQ1, FOSL1, NR2F2, KLF4 and TFCP2L1 as compared to a reference level
Determining in cancer cells, in particular tumor cells exposed to a chemotherapeutic agent, issued from a biological sample previously obtained from the subject the presence or the absence of cell that have a quantity of H3K27me3 lower than a reference level,
identifying cell as persistent cell when a cell overexpresses at least one genetic biomarker selected from the group comprising or consisting of LDHB, S100A2, KRT14, TAGLN, NNMT, FOXQ1, FOSL1, NR2F2, KLF4 and TFCP2L1, and having a quantity of H3K27me3 lower than a reference level which has been identified in the biological sample;
   and optionally classifying the subject as being at risk to have cancer recurrence when persister cells have been identified in the biological sample.

In a preferred embodiment of this method, persister cell overexpresses FOSL1 and KLF4, in particular at least FOXQ1, FOSL1, NR2F2 and KLF4, and more particularly at least FOXQ1, FOSL1, NR2F2, KLF4 and TFCP2L1.

In a preferred embodiment of this method persister cell overexpresses at least LDHB and S100A2.

In a preferred embodiment of this method persister cell overexpresses at least KRT14, TAGLN, and NNMT.

In another embodiment of the invention, it is provided a method for measuring the presence or absence of persister cells within a biological sample comprising cancer cells, in particular tumor cells exposed to a chemotherapeutic agent, issued from a biological sample previously obtained from at least one patient who has or had a cancer, wherein the method comprises:
Determining in cancer cell whether at least one genetic biomarker selected from the group comprising or consisting of S100A2, LDHB, KRT14, TAGLN, NNMT, FOXQ1, FOSL1, NR2F2, KLF4 and TFCP2L1 is over-expressed as compared to a reference level,
Identifying the presence of persister cell in the biological sample as cancer cell over-expressing at least one genetic biomarker, in particular all genetic biomarkers, which expression has been measured, or the absence of persister cells in the biological sample as no cancer cell over-expressing the genetic biomarker(s) which expression has been measured.

As indicated above, it is preferable to administer a different treatment, or to prolong treatment, or to associate new therapeutic treatment to one ongoing treatment, to a patient who has or had cancer and who is likely to develop cancer recurrence. Consequently, it is provided methods according to the invention for treating a patient who has or has cancer, and methods for determining which treatment should be administered to a subject who has or had cancer. Said treatment may in particular be a treatment aimed at blocking or slowing down the progress of the cancer, or reducing the likeliness of cancer recurrence, or aiming at blocking or killing or reducing persister cells within the subject.

Accordingly, it is provided a method for treating a human subject who had or has a cancer, said method comprising:
Determining in cancer cell, in particular tumor cells exposed to a chemotherapeutic agent, issued from a biological sample previously obtained from the subject whether at least one genetic biomarker selected from the group comprising or consisting of S100A2, LDHB, KRT14, TAGLN, NNMT, FOXQ1, FOSL1, NR2F2, KLF4 and TFCP2L1 is over-expressed as compared to a reference level,
Identifying the presence of persister cell in the biological sample as cancer cell over-expressing the one or more genetic biomarkers, in particular all genetic biomarkers, which expression has been measured,
When persister cell has been identified in the biological sample, administering to the subject an inhibitor of lysine demethylase or histone demethylase, in particular an inhibitor of lysine demethylase 6, more particularly of lysine demethylase 6 A/B or of H3K27me3 demethylase; and/or administering to the subject an inhibitor of $100A2, LDHB, KRT14, TAGLN, NNMT, FOXQ1, FOSL1, NR2F2, KLF4 or TFCP2L1 In a particular embodiment, it is provided a method for treating a human subject who had or has a cancer, said method comprising:

Determining in cancer cell, in particular tumor cells exposed to a chemotherapeutic agent, issued from a biological sample previously obtained from the subject whether at least one genetic biomarker selected from the group comprising or consisting of S100A2, LDHB, KRT14, TAGLN, NNMT, FOXQ1, FOSL1, NR2F2, KLF4 and TFCP2L1 is over-expressed as compared to a reference level,
Identifying the presence of persister cell in the biological sample as cancer cell over-expressing the one or more genetic biomarkers, in particular all genetic biomarkers, which expression has been measured,
When persister cell has been identified in the biological sample, administering to the subject an inhibitor of lysine demethylase or histone demethylase, in particular an inhibitor of lysine demethylase 6, more particularly of lysine demethylase 6 A/B or of H3K27me3 demethylase.

Indeed, the inventors found that the use of an inhibitor of lysine demethylase 6, which reduces the demethylation of H3K27me3, reduce the capability of cancer cells to become persister cells. By maintaining H3K27me3 level in persister cells at a sufficient level, cancer cells are less likely to differentiate into persister cells. The administration of an inhibitor of the demethylation of H3K27me3 in cancer cells, in particular in cancer cells exposed to chemotherapy, inhibit the emergence of persister cells. By preventing the emergence of persister cells, the risk to have cancer recurrence is reduced in patient treated with the inhibitor of lysine demethylase 6. In a particular embodiment of the invention, the inhibitor of lysine demethylase 6 is KDM 6A/B Lysine demethylase 6A/B inhibitor (KDM6A/Bi-GSK-J4). In a preferred embodiment, the inhibitor of lysine demethylase 6 is administered simultaneously with a chemotherapeutic agent, radiotherapy or immunotherapy agent, in particular with a chemotherapeutic agent, in particular a chemotherapeutic agent selected from the group comprising a thymidylate synthase (TS) inhibitor, more particularly fluorouacile (5-FU) or derivative thereof or analogue thereof.

According to another embodiment of the invention, it is provided a method for treating a human subject who had or has a cancer, said method comprising:

Determining in cancer cell, in particular tumor cells exposed to a chemotherapeutic agent, issued from a biological sample previously obtained from the subject whether at least one genetic biomarker selected from the group comprising or consisting of S100A2, LDHB, KRT14, TAGLN, NNMT, FOXQ1, FOSL1, NR2F2, KLF4 and TFCP2L1 is over-expressed as compared to a reference level,
Identifying the presence of persister cell in the biological sample as cancer cell over-expressing the one or more genetic biomarkers, in particular all genetic biomarkers, which expression has been measured,
When persister cell has been identified in the biological sample, administering to the subject an inhibitor of S100A2, LDHB, KRT14, TAGLN, NNMT, FOXQ1, FOSL1, NR2F2, KLF4 or TFCP2L1.

An inhibitor of S100A2, LDHB, KRT14, TAGLN, NNMT, FOXQ1, FOSL1, NR2F2, KLF4 or TFCP2L1 may correspond to a compound that inhibits the expression or the translation of genes encoding the listed biological markers, like but not limited to siRNA, antisense RNA, oligo nucleotides, or polypeptides and peptides which inhibit the function of the listed biological markers, like blocking antibodies, antagonist antibodies, functional equivalents of a native protein but lacking the means to correctly mimic the function of the native protein.

The administration of an inhibitor of S100A2, LDHB, KRT14, TAGLN, NNMT, FOXQ1, FOSL1, NR2F2, KLF4 or TFCP2L1 may be performed simultaneously with an ongoing therapy or cancer, like a conventional therapy of cancer by administration of a anti-cancer agent, like a chemotherapeutic agent or an immunotherapeutic agent, of by performing on the patient an anti-cancer treatment method, like particle therapy or radiotherapy, in particular proton-therapy. In some embodiments, the inhibitor of S100A2, LDHB, KRT14, TAGLN, NNMT, FOXQ1, FOSL1, NR2F2, KLF4 or TFCP2L1 is administered in combination with additional cancer therapies. In particular, inhibitor of S100A2, LDHB, KRT14, TAGLN, NNMT, FOXQ1, FOSL1, NR2F2, KLF4 or TFCP2L1 may be administered in combination with targeted therapy, immunotherapy such as immune checkpoint therapy and immune checkpoint inhibitor, co-stimulatory antibodies, chemotherapy and/or radiotherapy. As used herein, the term “antitumor chemotherapy” or “chemotherapy” has its general meaning in the art and refers to a cancer therapeutic treatment using chemical or biochemical substances, in particular using one or several antineoplastic agents or chemotherapeutic agents. As used herein, the term “immunotherapy” refers to a cancer therapeutic treatment using the immune system to reject cancer. The therapeutic treatment stimulates the patient's immune system to attack the malignant tumor cells. Suitable examples of radiation therapies include, but are not limited to external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like).

In a particular embodiment of the invention, it is provided a method for treating a human subject who had or has a cancer, said method comprising:
Determining in cancer cell, in particular tumor cells exposed to a chemotherapeutic agent, issued from a biological sample previously obtained from the subject whether at least one genetic biomarker selected from the group comprising or consisting of S100A2, LDHB, KRT14, TAGLN, NNMT, FOXQ1, FOSL1, NR2F2, KLF4 and TFCP2L1 is over-expressed as compared to a reference level,
Identifying the presence of persister cell in the biological sample as cancer cell over-expressing the one or more genetic biomarkers, in particular all genetic biomarkers, which expression has been measured,
When persister cell has been identified in the biological sample, administering to the subject an inhibitor of at least one biological marker over expressed by the persister cells, and in particular an inhibitor for each biological marker over expressed by persister cells, said inhibitor being selected among the inhibitors of S100A2, LDHB, KRT14, TAGLN, NNMT, FOXQ1, FOSL1, NR2F2, KLF4 or TFCP2L1.

In a preferred embodiment, it is further administered to the subject an inhibitor of lysine demethylase 6, more particularly of lysine demethylase 6 A/B or of H3K27me3 demethylase, and/or a chemotherapeutic agent, radiotherapy or an immunotherapeutic agent, in particular a chemotherapeutic agent selected from the group comprising a thymidylate synthase (TS) inhibitor, more particularly fluorouacile (5-FU) or derivative thereof or analogue thereof.

The present invention also concerns a method for treating or preventing cancer recurrence in a patient who had or has cancer, and comprising:
providing a biological sample from the subject;
determining the presence of persistent cells in the sample according to any embodiment disclosed herein;
determining if the patient is likely to have cancer recurrence, and when the patient is likely to have cancer recurrence or when persister cells are present within the biological sample;
administering to the patient at least one therapeutic agent selected among the group comprising or consisting of:
   a chemotherapeutic agent, radiotherapy or an immunotherapeutic agent, in particular a chemotherapeutic agent selected from the group comprising a thymidylate synthase (TS) inhibitor, more particularly fluorouacile (5-FU) or derivative thereof or analogue thereof; and/or
   at least one inhibitor of S100A2, LDHB, KRT14, TAGLN, NNMT, FOXQ1, FOSL1, NR2F2, KLF4 or TFCP2L1; and/or
   an inhibitor of lysine demethylase 6, more particularly of lysine demethylase 6 A/B or of H3K27me3 demethylase.

The present invention also concerns a method for treating a patient identified as having a cancer, of who had a cancer which is under complete or partial remission, and comprising:
providing a biological sample from the subject;
determining the presence of persistent cells in the sample according to any embodiment disclosed herein;
when persistent cells are present within the sample, administering to the patient at least one therapeutic agent selected among the group comprising or consisting of:
   a chemotherapeutic agent, radiotherapy or an immunotherapeutic agent, in particular a chemotherapeutic agent selected from the group comprising a thymidylate synthase (TS) inhibitor, more particularly fluorouacile (5-FU) or derivative thereof or analogue thereof; and/or
   at least one inhibitor of S100A2, LDHB, KRT14, TAGLN, NNMT, FOXQ1, FOSL1, NR2F2, KLF4 or TFCP2L1; and/or
   an inhibitor of lysine demethylase 6, more particularly of lysine demethylase 6 A/B or of H3K27me3 demethylase.

The present invention also concerns a method for reducing the number of persister cells upon chemotherapy exposure comprising administering to a patient in need thereof an effective amount of an inhibitor of lysine demethylase, in particular an inhibitor of lysine demethylase 6, more particularly of lysine demethylase 6 A/B or of H3K27me3 demethylase, said method comprising the determination of the presence of persister cells in the patient according to any embodiment disclosed herein before administering to the patient the inhibitor of lysine demethylase.

The present invention also concerns a method for reducing the number of persister cells upon chemotherapy exposure comprising administering to a patient in need thereof an effective amount of a chemotherapeutic agent, radiotherapy or an immunotherapeutic agent, in particular a chemotherapeutic agent selected from the group comprising a thymidylate synthase (TS) inhibitor, more particularly fluorouacile (5-FU) or derivative thereof or analogue thereof, said method comprising the determination of the presence of persister cells in the patient according to any embodiment disclosed herein before administering to the patient a chemotherapeutic agent, radiotherapy or an immunotherapeutic agent, in particular a chemotherapeutic agent selected from the group comprising a thymidylate synthase (TS) inhibitor, more particularly fluorouacile (5-FU) or derivative thereof or analogue thereof.

The present invention also concerns a method for reducing the number of persister cells upon chemotherapy exposure comprising administering to a patient in need thereof an effective amount of one inhibitor of S100A2, LDHB, KRT14, TAGLN, NNMT, FOXQ1, FOSL1, NR2F2, KLF4 or TFCP2L1, said method comprising the determination of the presence of persister cells in the patient according to any embodiment disclosed herein before administering to the patient the inhibitor.

The present invention also concerns a method for determining if persister cells are present in a patient having a cancer and who is being treated against said cancer or who is resistant to a treatment against said cancer or who is receiving a treatment that is likely to induce differentiation of tumor cells into persister cells, the method comprising
Determining in cancer cell, in particular tumor cells exposed to a chemotherapeutic agent, issued from a biological sample previously obtained from the patient whether at least one genetic biomarker selected from the group comprising or consisting of LDHB, S100A2, KRT14, TAGLN, NNMT, FOXQ1, FOSL1, NR2F2, KLF4 and TFCP2L1 is over-expressed as compared to a reference level,
Identifying the presence of persister cell in the biological sample as cancer cell over-expressing at least one genetic biomarker, in particular all genetic biomarkers, which expression has been measured.

The invention also concerns the use of one or more genetic biomarkers selected from the group comprising or consisting of S100A2, LDHB, KRT14, TAGLN, NNMT, FOXQ1, FOSL1, NR2F2, KLF4 and TFCP2L1, for in vitro assessing if a cancer cell is a persister cell.

The invention also concerns an inhibitor of LDHB, KRT14, TAGLN, NNMT, FOXQ1, FOSL1, NR2F2, KLF4 or TFCP2L1 for use in the treatment of a cancer wherein said cancer exhibits/comprises persister cells. In particular, the cancer is a breast cancer, and more particularly a TNBC. In a particular embodiment, the inhibitor off LDHB, KRT14, TAGLN, NNMT, FOXQ1, FOSL1, NR2F2, KLF4 or TFCP2L1 is used in combination with a conventional treatment of cancer, as disclosed herein. Conventional treatment of cancer includes the administration of anti-cancer agent, like but not limited to chemotherapeutic agent and immunotherapeutic agent. Conventional treatment also includes treating a patient with radiotherapy or particle therapy as disclosed herein.

The invention also concerns a method for identifying biological markers expressed or over-expressed by persister cells associated to a particular type of cancer. Accordingly, such a method comprises:
Providing a biological sample comprising cancer cells previously obtained from a patient having a cancer, in particular a tumor sample,
Generating a Patient-Derived Xenograft (PDX) by associating cells cancer issued from the biological sample to receiving animals,
Treating a first group of receiving animals PDX within the receiving animal with a therapeutic agent, in particular by administering a chemotherapeutic agent or an immunotherapeutic agent, or by a therapeutic method, in particular by radiotherapy or particle therapy, more particularly by proton therapy, thereby leading to treated tumor cells,
Optionally, treating a second group of receiving animals with a control agent, thereby leading to control tumor cells,
Recover treated tumor cells from the receiving animals, and when applicable control tumor cells,
Sequencing the RNA of treated tumor cells, in particular by single-cell RNA-sequencing,
Determining the biological markers expressed, in particular over-expressed, by treated tumor cells by comparing their phenotype with the phenotype of control cells, in particular with phenotype of control tumor cells.

Tumor cells issued from the treated PDX are cells which are likely to become persistent and lead to cancer recurrence. By analysing the phenotype profile of these cells, for example by quantifying RNA transcripts issued from these cells, and comparing this profile with control cells, specific biological marker(s) of persister cells associated with a particular type of cancer can be determined. The different steps of the method may be performed according to the example of the invention wherein this method is performed starting from samples issued from TNBC-patients.

In accordance with a complementary aspect of the invention, the application relates to products or reagents for the detection and/or determination and/or measurement of the levels of expression of said selected biological markers, and to manufactured articles, compositions, pharmaceutical compositions, kits, tubes or solid supports comprising such reagents, as well as to computer systems (in particular, computer program product and computer device), which are specially adapted to carrying out a method of the invention. The present invention also concerns kit for performing any method described herein. The application is in particular relative to a reagent which specifically detects a transcription product (RNA) of at least one of, in particular each of, said biological marker selected from said list of ten biological marker of the invention, or a translation product of at least one of, in particular each of, said biological marker selected from said list often biological markers of the invention (protein, or post-translational form of this protein, such as a specific fragment of this protein). The application is in particular relative to a reagent which specifically detects the quantity of H3K27me3, combined or not with the reagent which specifically detects a transcription product (RNA) of at least one of, in particular each of, said biological marker selected from said list of ten biological marker of the invention, or a translation product of at least one of, in particular each of, said biological marker selected from said list often biological markers of the invention (protein, or post-translational form of this protein, such as a specific fragment of this protein). Advantageously, a set of such reagents is formed which detects each of said transcription products of said selected biological markers and/or which detects each of said translation products of said biological markers selected from said list of ten biological markers of the invention, i.e. a set of reagents which specifically detects at least one expression product for each of these ten biological markers. Preferably, said reagents not only specifically detect a transcription or translation product, but can also quantify it. More advantageously, the set further comprises a reagent for quantifying H3K27me3.

Said reagents may, for example, hybridize specifically to the RNA of said selected genes and/or to the cDNA corresponding to these RNAs (under at least stringent hybridization conditions), or bind specifically to proteins encoded by said selected genes (or to specific fragments of these proteins), for example in an antigen-antibody type reaction. Said reagents of the invention may in particular be: nucleic acids (DNA, RNA, mRNA, cDNA), including oligonucleotide aptamers, optionally tagged to allow them to be detected, in particular with fluorescent tags which are well known to the skilled person, or protein ligands such as proteins, polypeptides or peptides, for example aptamers, and/or antibodies or fragments of antibodies.

### EXAMPLES ILLUSTRATING THE INVENTION

### Material and Method

### Experimental Approaches:

Patient samples and PDX models. Patient samples used in this study (n=9) originated from patient treated at Institut Curie with residual triple-negative breast cancers post-neoadjuvant chemotherapy, who gave informed consent for the profiling. In this study, we used three xenograft models generated from three different residual triple-negative breast cancers post-neoadjuvant chemotherapy (HBCx95 called PDX_95, HBCx39 called PDX_39 and HBCx172 called PDX_172 in the manuscript, see Extended Table2) established previously at Curie Institute with informed consent from the patient^{46,47}. Female Swiss nude mice were purchased from Charles River Laboratories and maintained under specific-pathogen-free conditions. Mouse care and housing were in accordance with institutional guidelines and the rules of the French Ethics Committee (project authorization no. 02163.02).

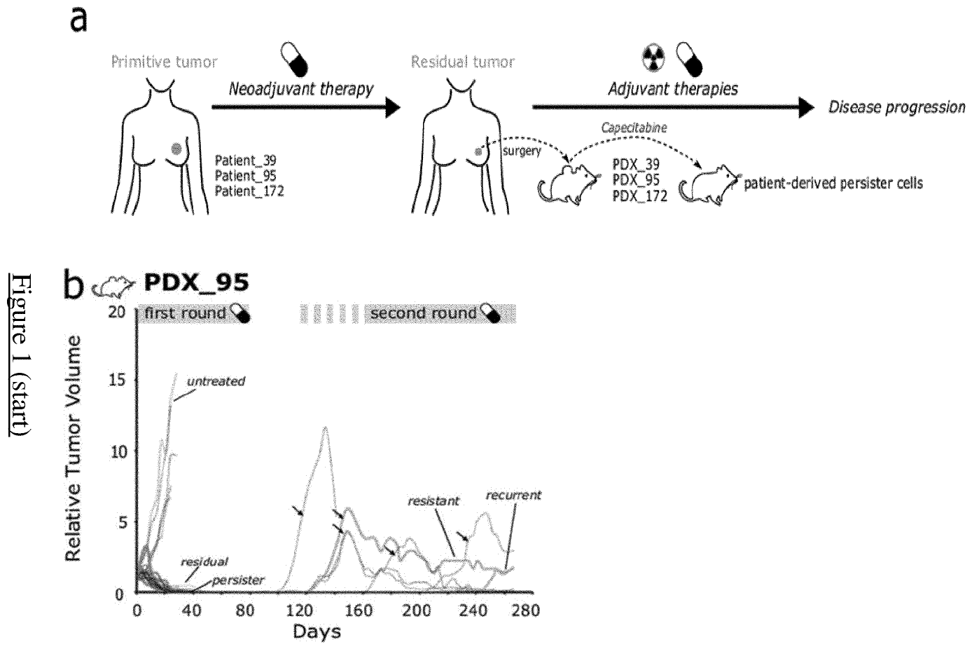

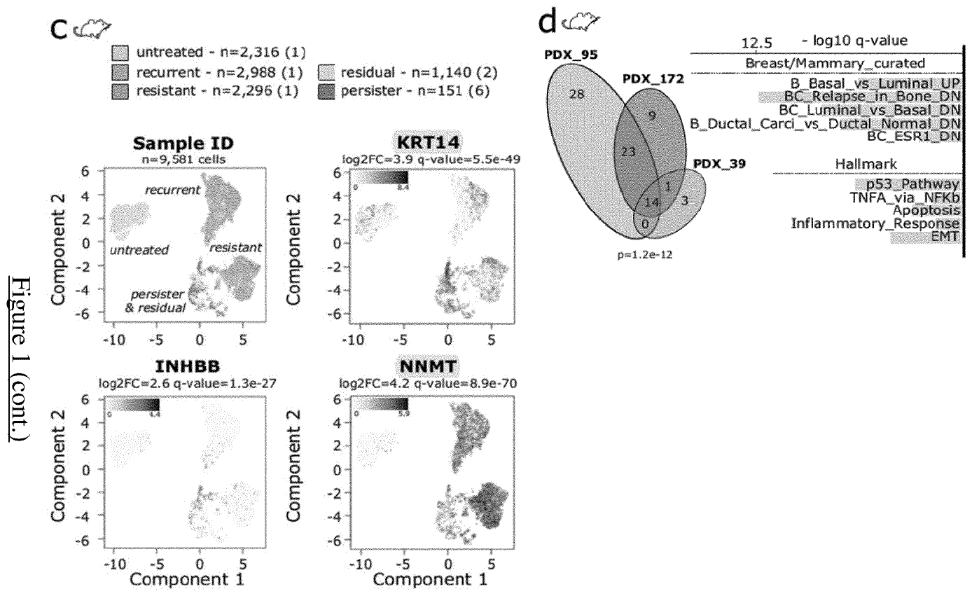

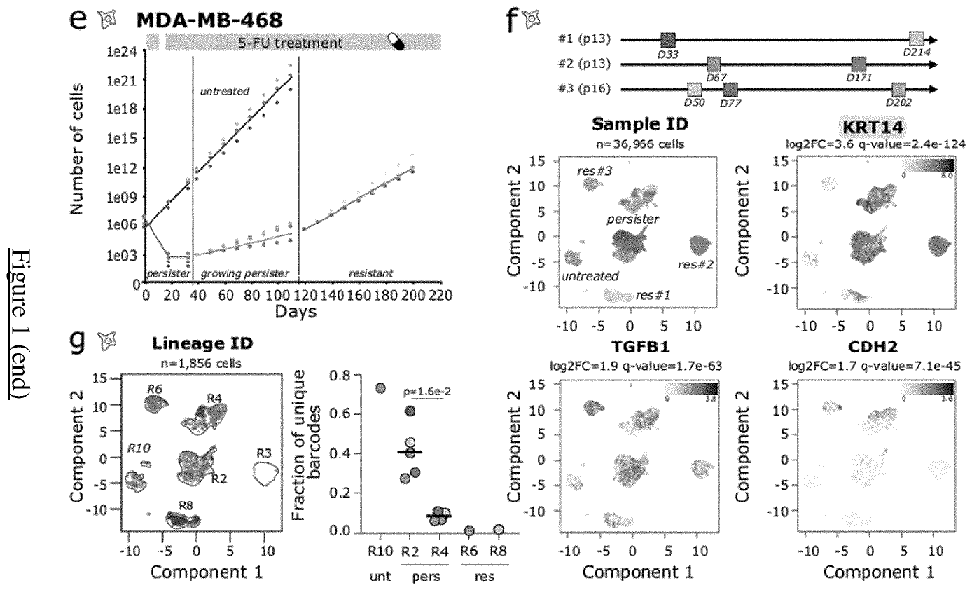

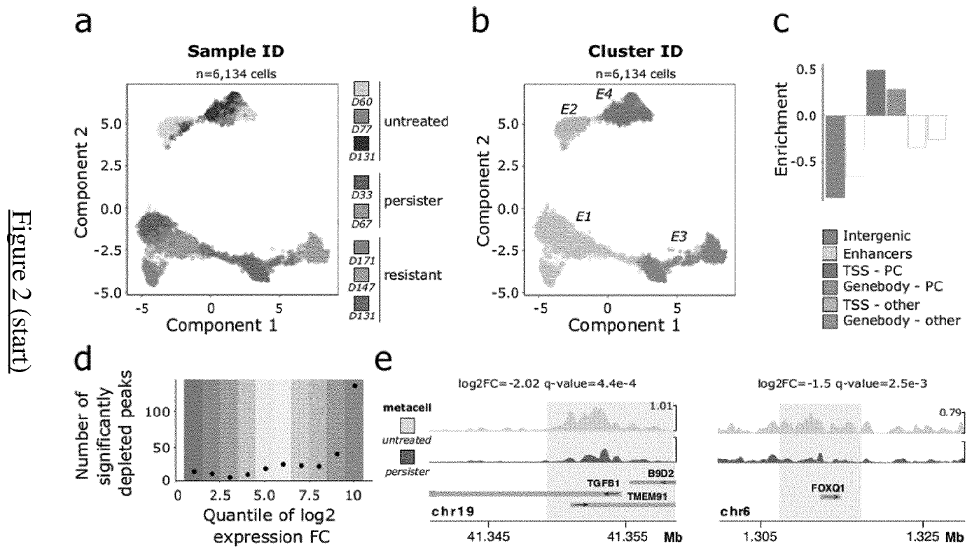

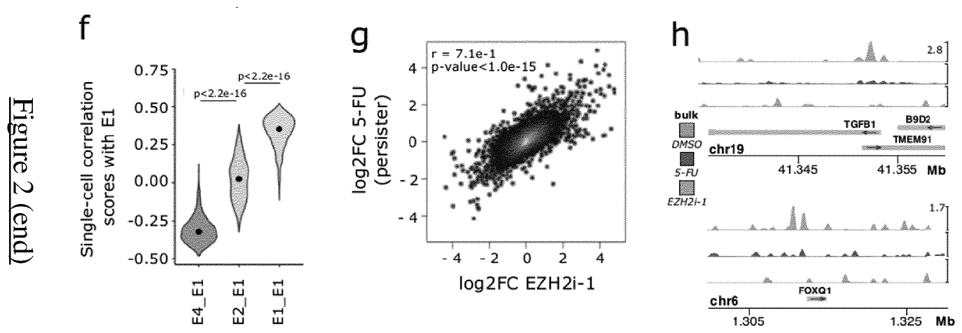

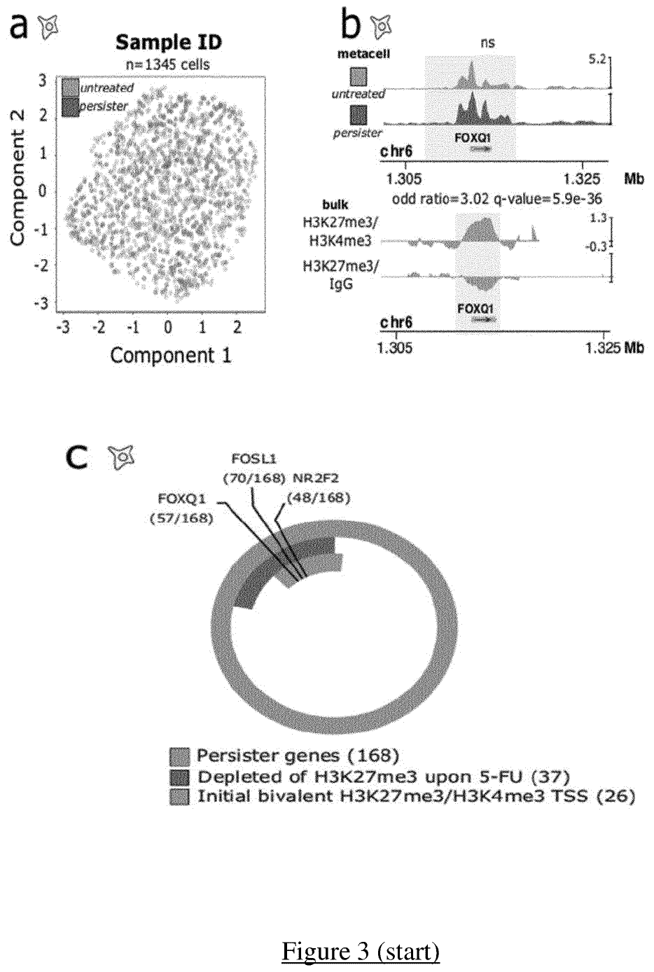

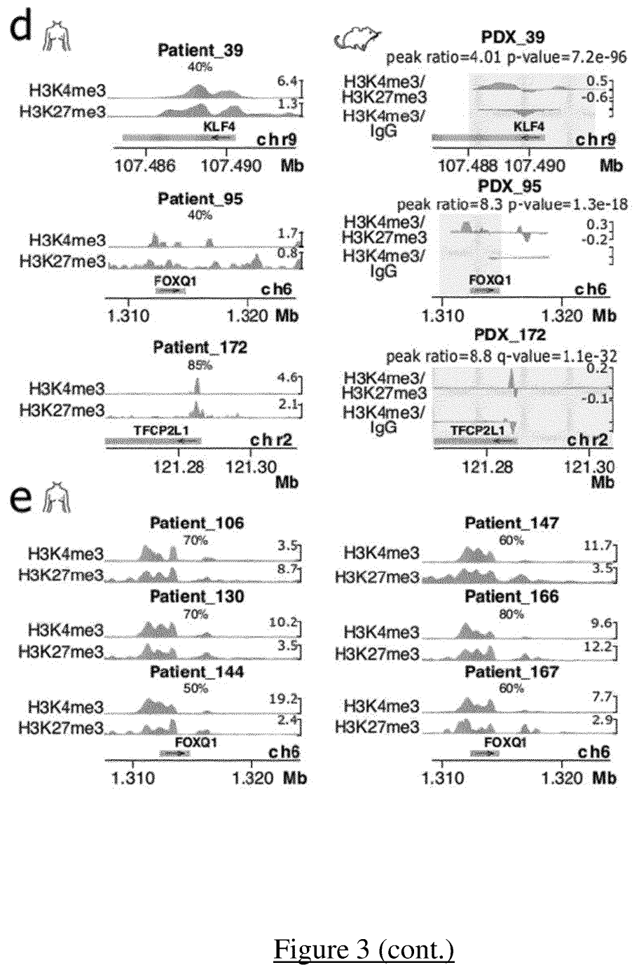

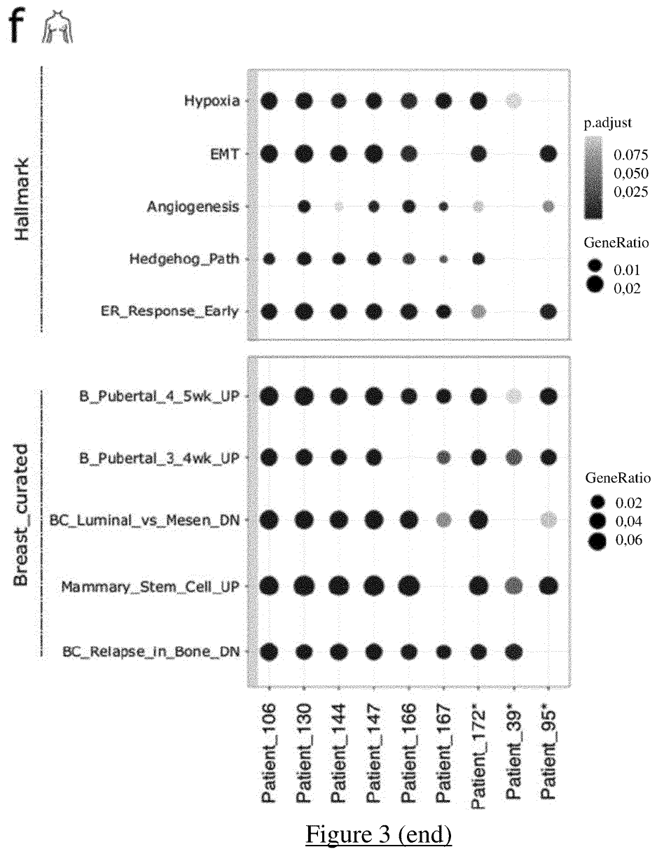

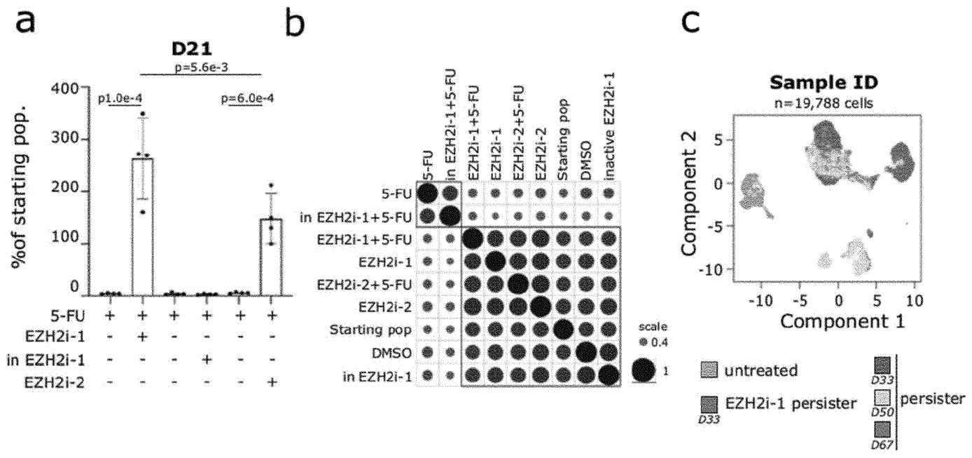

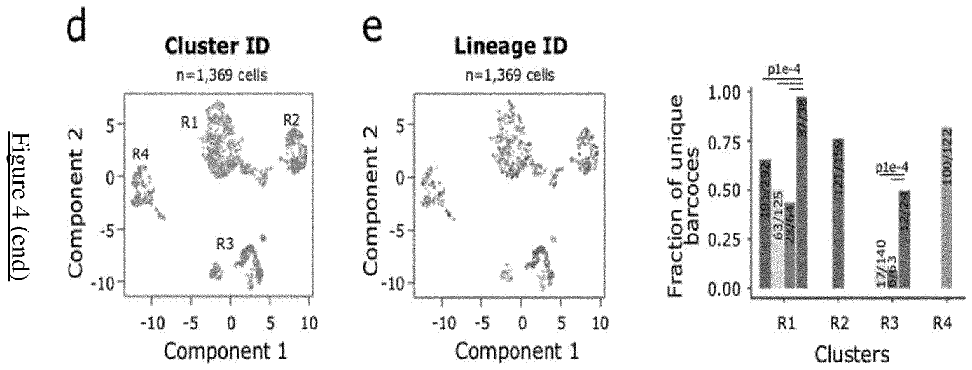

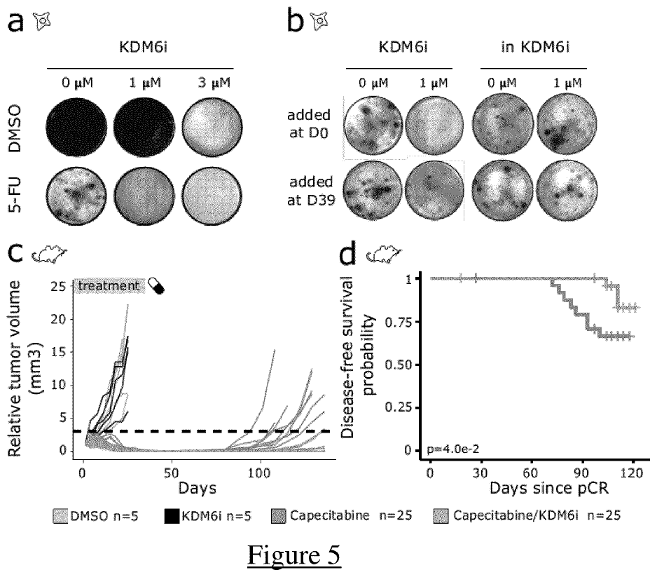

FIG. 1b: Five mice were not treated and kept as controls (termed “untreated”) and twenty-seven mice were treated orally with Capecitabine (Xeloda; Roche Laboratories) at a dose of 540 mg/kg, 5 d/week for 6 to 14 weeks. Relative tumor volumes (mm3) were measured as described previously¹⁸. Eight mice were sacrificed after the first round of chemotherapy to study “residual” tumors (2 mice) or “persister” (6 mice) human tumor cells. Seven mice with “recurrent” tumors (tumor volume between 200 and 600 mm3) were treated with a second round of Capecitabine to which they responded or not. “Resistant” refers to a tumor which maintains a constant volume under this second round of treatment.

Extended FIG. 1f: Three mice were not treated and kept as controls (termed “untreated”) and fourteen mice were treated orally with Capecitabine at a dose of 540 mg/kg, 5 d/week for 7 weeks and sacrificed to study “persister” human tumor cells.

Extended FIG. 1l: Six mice were not treated and kept as controls (termed “untreated”) and four mice were treated orally with Capecitabine for 7 weeks and sacrificed to study “persister” human tumor cells.

FIG. 5c: Five mice were treated intraperitoneally with DMSO, five mice were treated intraperitoneally with GSK-J4 alone at a dose of 50 mg/kg, 5 d/week for 25 days. Twenty-five mice were treated orally with Capecitabine at a dose of 540 mg/kg, 5 d/week for 36 days and twenty-five mice were co-treated with Capecitabine and GSK-J4 for 36 days. Tumor volumes (mm3) were measured to follow recurrence. FIG. 5d: Disease-free survival was defined as the number of days between the observation of a complete response (relative tumor volume RTV compared to volume at onset of treatment<0.2) after the first round of Capecitabine treatment, and the appearance of a recurrent tumor (RTV>3). Statistical analysis was performed using a log-rank test.

Before downstream analysis (scChIP-seq, scRNA-seq or sequential ChIP-seq), control and treated tumors were digested for 2 h at 37° C. with a cocktail of Collagenase I (Roche, Ref: 11088793001) and Hyaluronidase (Sigma-Aldrich, Ref: H3506). Cells were then individualized at 37° C. using a cocktail of 0.25% Trypsin-Versen (Thermo Fisher Scientific, Ref: 15040-033), Dispase II (Sigma-Aldrich, Ref: D4693) and DNase I (Roche, Ref: 11284932001) as described previously⁴⁸. Then, eBioscience red blood cell lysis buffer (Thermo Fisher Scientific, Ref: 00-4333-57) was added to the cell suspension to remove red blood cells. To increase the viability of the final cell suspension, dead cells were removed using the Dead Cell Removal Kit (Miltenyi Biotec, Ref: 130-090-101).

Cell lines, culture conditions and drug treatments. MDA-MB-468 cells were cultured in DMEM (Gibco-BRL, Ref: 11966025), supplemented with 10% heat-inactivated fetal calf serum (Gibco-BRL, Ref: 10270-106). HCC38 and BT20 cell lines were cultured in RPMI 1640 (Gibco-BRL, Ref: 11875085), supplemented with 10% heat-inactivated fetal calf serum. All cell lines were cultured in a humidified 5% CO2 atmosphere at 37° C., and were tested as mycoplasma negative. GSKJ4 (KDM6A/B inhibitor, Sigma, Ref: SML0701), GSKJ5 (GSK-J4 inactive isomer, Abcam, Ref: ab144397), UNC1999 (EZH2 inhibitor, Abcam, Ref: ab 146152), UNC 2400 (UNC 1999 inactive isoform, Tocris, Ref: 4905) and GSK126 (EZH2 inhibitor, Sigma, Ref:) were used at indicated concentrations. Cells were treated with 5 μM of 5-FU (Sigma, Ref: F6627) alone or in combination with KDM6A/Bi or EZH2i for indicated days. For EZH2i, cells were pretreated with UNC 1999, UNC 2400 or GSK 126 for 10 days before the addition of 5-FU for an additional 21 days (FIG. 4 and Extended FIG. 8).

Colony forming assay. TBNC cells were plated in 6 multi-well plates at a density of 200,000 cells per well and treated with the indicated drugs for 60 days (MDA-MB-468, FIG. 5a/b and Extended FIG. 9b) or 56 days (BT20) or 50 days (HCC38) (Extended FIG. 9). Cultures were incubated in humidified 37° C. incubators with an atmosphere of 5% CO2 in air, and treated plates were monitored for growth using a microscope. At the time of maximum foci formation, colony formation was evaluated after a staining with 0.5% Crystal Violet (Sigma, ref: C 3886).

Cell proliferation, doubling time and IC50. MDA-MB-468, HCC38 and BT20 cells were stained with Trypan Blue (Invitrogen, Ref: T10282) exclusion test, and counted using a Countess automated cell counter (Invitrogen, Ref: C10228) at indicated time of treatment (FIG. 4a and Extended FIG. 8a/d/f).

Doubling time (Extended FIG. 2b) was calculated using this formula:

$$“ DoublingTime = duration * log ( 2 ) / ( log ( Final Concentration ) - log ( Initial Concentration ) ) ”$$

For untreated condition and resistant condition, cell numbers were evaluated on cell population during 10 days (n=3). For persister condition, cells were counted manually under the microscope at day 13 and day 30 of treatment. Doubling time of 5-FU growing persister cells was studied from single cell to confluent colony by assaying cell number every 4 days during 27 days (n=6 single cells).

MDA-MB-468 untreated and chemoresistant cells were plated in 96 multi-well plates at a density of 10,000 cells per well and treated with increased concentration of 5-FU (1 μM to 0.5M) for 72 h. Cell cytotoxicity was assayed with XTT kit (Sigma, Ref: 11465015001) and IC 50 was calculated as the concentration of 5-FU that is required to obtain 50% of cell viability (Extended FIG. 2b).

Cells were classified as ‘persister’, ‘growing persister’ or ‘resistant’ based on a combination of 2 biological markers: (i) doubling time under 5-FU, (ii) IC50 to 5-FU (Extended FIG. 2b, FIG. 1e):
‘persister’ correspond to non-dividing cells (infinite doubling time)
‘growing persister’ are dividing cells with a doubling time significantly higher than resistant cells under 5-FU
‘resistant’ correspond to cells with a doubling time comparable to untreated cells and a significant higher IC 50 to 5-FU compared to untreated cells

It should be noted that due to low number of persister cells (0.01% of initial population) and growing persister cells, we could not measure the IC50 to 5-FU of these two states.

For Extended FIG. 9 d cells were treated with 5-FU at 5 uM and indicated concentrations of GSK-J4 or GSK-J5 (D-5 indicated 5 days pre-treatment with GSK-J4 or GSK-J5 before 5-FU treatment (D0), D0 indicated co-treatment 5-FU and GSK-J4 or GSK-J5, D10 and D30 indicated that treatment with GSK-J4 or GSK-J5 started 10 days or 30 days respectively after the onset of 5-FU treatment (D0). The number of persister cells were counted manually under the microscope at day 42 (n=3).

The GraphPad PRISM 9 was used for statistics and the results represent the mean±sd of three independent experiments. Statistical analysis was performed using the Bonferroni test for multiple comparisons between samples (FIG. 4a, Extended FIG. 8a/d/f, Extended FIG. 9b/d and Extended FIG. 2b—right) or one-tailed Mann-Whitney test for the comparison between two conditions (Extended FIG. 2b—left).

Western blotting. In Extended FIG. 8b/e/g, DMSO- and EZH2i-treated cells were lysed at 95° C. for 10 minutes in Laemmli buffer (50 mM Tris-HCl [pH 6.8], 2% SDS, 5% glycerol, 2 mM DTT, 2.5 mM EDTA, 2.5 mM EGTA, 4 mM Sodium Orthovanadate, 20 mM Sodium Fluoride, protease inhibitors, phosphatase inhibitors) and proteins concentrations were measured using a Pierce BCA protein Assay Kit (Thermo Fisher Scientific, Ref: 23225/23227). 10 μg of proteins were then separated on a 4-15% Mini-PROTEAN TGX Stain-Free Gel (Bio-Rad, Ref: 4568085) at 160V. After transfer, the membrane was blocked for 1 h at room temperature in PBS pH 7.4 containing 0.1% Tween-20 and 1% milk (Regilait). Incubation anti-H3K27me3 (Dilution: 1:2000, Cell Signaling, Ref: 9733) or EZH2 (Dilution: 1:2000, Cell Signaling, Ref: 5246) or Tubulin (Dilution: 1:1000, Thermo Fisher Scientific, Ref: 31460) primary antibodies diluted in PBS pH 7.4, 0.1% Tween-20 were performed at 4° C. overnight. Following 2 h incubation at room temperature with an anti-rabbit or mouse peroxidase-conjugated secondary antibody (Dilution: 1:10000, Thermo Fisher Scientific, Ref: 31460 or Ref: 31430) diluted in PBS pH 7.4, 0.1% Tween-20, antibody-specific labeling bands were revealed (Bio-Rad, ChemiDoc MP) using a SuperSignal West Pico PLUS Chemiluminescent Substrate (Thermo Fisher Scientific, Ref: 34579).

Lentivirus packaging and cell transduction. Lentivirus was produced by transfecting the barcode plasmids pRRL-CMV-GFP-BCv2Ascl and p8.9-QV and pVSVG into HEK293T cells as previously described³⁰. MDA-MB-468 cells from ATCC were infected at passage 11 with lentivirus produced from the barcode library (pRRL-CMV-GFP-BCv 2Ascl) which includes 18206 different barcodes of 20 bp of a random stretch, at a low multiplicity of infection (MOI 0.1) to minimize the number of cells marked by multiple barcodes. Three weeks after transduction, cells were sorted for GFP expression to select cells with barcode insertion, and used for drug treatment.

Single-cell RNA-seq. For each single cell suspension (DMSO-DO- #1, 5-FU-D33- #1, 5-FU-D214- #1, 5-FU-D67- #2, 5-FU-D171- #2, 5-FU-D50- #3, 5-FU-D77- #3 and 5-FU-D202- #3) or PDX dissociated cells (PDX_95, PDX_39 or PDX_172, untreated and persister cells), approximately 3,000 cells were loaded on a Chromium Single Cell Controller Instrument (Chromium Single Cell 3′v3, 10× Genomics, Ref: PN-1000075) according to the manufacturer's instructions. Samples and libraries were prepared according to the manufacturer's instructions. Libraries were sequenced on a NovaSeq 6000 (Illumina) in PE 28-8-91 with a coverage of 50,000 reads/cell.

Bulk lineage barcode library preparation and sequencing. Lineage barcodes are recovered by isolating genomic DNA from cells of interest (NucleoSpin Tissue, Mini kit for DNA from cells and tissue, Macherey Nagel, Ref: 740952.50). From the isolated genomic DNA, barcodes are amplified with three nested PCR steps as decribed in³⁰ (see Extended Table 1 for primer sequence). In short, after a first specific PCR for the common region of the lineage barcodes, the amplified material was prepared for sequencing by addition of the illumina sequencing adaptaters and indexing and purification. Sequencing was done in order to obtain 50 reads, on average, per barcoded cell.

Bulk ChIP-seq. ChIP experiments were performed as previously described¹⁶ on 3×10⁶ MDA-MB-468 cells (DMSO-D67- #2, DMSO-D77- #3, DMSO-D113- #4, 5-FU-D67- #2, 5-FU-D77- #3, 5-FU-D113- #4) using an anti-H3K27me3 antibody (Cell Signaling Technology, Ref: 9733-C36B11). Sequencing libraries were prepared using the NEBNext Ultra II DNA Library Prep Kit (NEB, Ref: E7645S) according to the manufacturer's instructions. Libraries were sequenced on a NovaSeq 6000 (Illumina) in SE50 mode.

Single-cell Chip-seq. Cells (dmso-d60- #1, Dmso-d77- #3, Dmso-d131- #5, 5-FU-D33- #1, 5-FU-D67- #2, 5-FU-D171- #2, 5-FU-D147- #3, 5-FU-D131- #6) were labeled by 15 min incubation with 1 μM CFSE (CellTrace CFSE, ThermoFisher Scientific, Ref: C34554). Cells were then resuspended in PBS supplemented with 30% Percoll, 0.1% Pluronic F68, 25 mM Hepes pH 7.4 and 50 mM NaCl. Cell encapsulation, bead encapsulation and 1:1 droplet fusion was performed as previously described¹⁶, see Extended Table 1 for the sequence of bead barcodes. Immunoprecipitation with H3K27me3 antibody (Cell signaling, Ref: 9733-C36B11) or H3K4me3 antibody (Cell signaling, Ref: 9751-C42D8), DNA amplification and library were performed as in¹⁶. Libraries were sequenced on a NovaSeq 6000 (Illumina) in PE100, with 4 dark cycles on Read 2, with a coverage of 100,000 reads/cell.

Quantitative chromatin profiling with chromatin indexing. Chromatin isolation, indexing, immunoprecipitation and library preparation was adapted from⁴⁹. Briefly, 50,000 MDA-MB-468 were lysed and digested with MNase for 20 min at 37° C. in the following buffer: 46 mM Tris-HCl pH 7.4, 0.154M NaCl, 0.1% Triton, 0.1% NaDoc, 4.65 mM CaCl2, 0.47× Protease Inhibitor Cocktail (Roche, Ref: 11873580001) and 0.09 u/uL MNase (Thermo Scientific, Ref: EN 0181). Fragmented nucleosomes were then ligated for at least 24 h at 16° C. to double-stranded barcoded adapters containing 8 bp barcodes to combine samples: Pac1-T7-Read2-8bpBarcode-linker-Pac1 (Extended Table 1). Next, 5 indexed chromatin samples (DMSO, 5-FU, UNC, 5-FU+UNC, GSK-J4) were pooled, each containing a different 8-bp barcode, to perform anti-H3K27me3 ChIP (Cell Signaling, Ref: 9733-C36B11) on 250,000 cells in total in each pool. ChIP and DNA amplification was carried out as for scChIP-seq¹⁶ and a sequencing library was produced for both IP and input pools and sequenced on NovaSeq 6000 (Illumina) in PE100 mode.

Sequential ChIP-seq. Primary ChIP experiments were performed as described previously¹⁶ on 10×10⁶ untreated MDA-MB-468, BT20 or HCC38 cells or untreated PDX_95, PDX_39 or PDX_172 tumor dissociated cells using the anti-H3K27me3 antibody (Cell Signaling, Ref: 9733-C36B11-MDA-MB-468) or anti-H3K4me3 antibody (Cell Signaling, Ref: 9751-C42D8-MDA-MB-468-bis, BT20, HCC38 and PDX models). After washes, samples were eluted twice at 37° C. for 15 min under agitation in an elution buffer (50 mM Tris-Hcl pH8, 5 mM EDTA, 20 mM DTT, 1% SDS) as in. Samples were diluted 10 times to decrease SDS and DTT concentration. 10% of the eluted chromatin was kept as primary ChIP. Secondary ChIP, re-ChIP, was performed overnight on the rest of the primary immuno-precipitated chromatin using an anti-H3K4me3 antibody (MDA-MB-468) or anti-H3K27me3 (MDA-MB-468-bis, BT20, HCC38 and PDX models) or using an anti-IgG antibody (Cell signaling, Ref: 3900—all samples) as a control, to determine the background level of the re-ChIP experiment. After washes, samples were eluted twice at 65° C. for 15 min under agitation in 0.1 M NaHCO3 and 1% SDS as in⁵⁰. After reverse crosslinking and DNA clean-up, 3 to 15 ng of immunoprecipitated DNA were used to prepare the sequencing libraries using the NEBNext Ultra II DNA Library Prep Kit (NEB, Ref: E7645S) according to the manufacturer's instructions. Libraries were sequenced on a NovaSeq 6000 (Illumina) in SE100 mode. For MDA-MB-468, we verified that the two ways (H3K27me3->H3K4me3 and H3K4me3->H3K27me3) yielded similar results. We found a significant overlap of the 1,547 and 2,490 bivalent genes obtained with the two ways (p=2.2e−16, Ext. FIG. 6g) and found that the enriched pathways were strongly correlated (Pearson's r=0.81, Ext. FIG. 6h).

CUT&Tag on frozen tumor samples. CUT&Tag was performed as in Kaya-Okur et al. with minor modifications on 50,000 to 100,000 nuclei with 1:50 antibody (Cell Signaling Antibodies: Anti-H3K27me3, Ref: 9733-C36B11, Anti-H3K4me3, Ref: 9751-C42D8)^{17,51}. All washes were performed in a volume of 500 μL and all centrifugations were done using a swinging bucket centrifuge at 1300 g, 4 min, at 4° C. for nuclei preparation and 600 g, 8 min, 4° C. for subsequent steps. Nuclei were extracted and permeabilized from 10-20 mg frozen tumor tissues by incubating samples 10 min on ice in 6 mL ice-cold NE1 buffer (20 mM HEPES pH7.2 , KCl 10 mM, spermidine 0.5 mM, glycerol 20%, BSA 1%, NP-40 1%, digitonin 0.01%, proteases inhibitor 1>) after mechanical dissociation. Following antibody incubation and tagmentation, samples were incubated for 1 h at 55° C. with max speed agitation with 3 uL SDS 10% and 2.5 uL 20 mg/mL proteinase K. After DNA extraction (Qiagen, Ref: 139046 MaXtract High density), PCR amplification (with 17 cycles, 20 s at 63° C. combined annealing/extension step) of the sequencing libraries was performed and profiles were checked on the Agilent TapeStation using High-sensitivity D1000 reagents. CUT&Tag libraries were sequenced on a NovaSeq 6000 (Illumina) in PE50 mode.

Whole exome sequencing. Genomic DNA from samples (DMSO-D0, DMSO-D147- #3, DMSO-D171- #5, DMSO-D131- #6, 5-FU-D67- #2, 5-FU-D153- #2, 5-FU-D50- #3, 5-FU-D147- #3, 5-FU-D171- #5 and 5-FU-D131- #6) were extracted with NucleoSpin Tissue, Mini kit for DNA from cells and tissue (Macherey Nagel, Ref: 740952.50) and sequenced on a NovaSeq 6000 (Illumina) with a 100× depth.

### Computational Approaches

Single-cell RNA-seq analysis. The scRNA-seq sequencing files were preprocessed using the cellRanger pipeline. For PDX samples, files were aligned against hg19 and mm10 genomes and only cells with a majority of human reads were retained for the analysis. For the MDA-MB-468 human cell line, sequences were aligned against the hg38 genome only. Cells with less than 3,000 cells for MDA-MB-468 or 2,500 for PDX or more than 8,000 detected genes, or more than 100,000 reads were filtered out, as well as cells with a percent of mitochondrial reads greater than 15% or a percentage of spike in greater than 5%. Normalization, dimensionality reduction and Louvain clustering was done using monocle3 (v0.2.2)⁵² keeping the first 50 Principal Components (PC). Cell cycle was determined for each cell using Seurat (v3.1.5)⁵³. For MDA-MB-468 datasets, we removed from subsequent analysis clusters with less than 0.5% of the total cells (150 cells) (clusters R1, R5, R7, R9, R11 and R12). Differentially expressed genes were obtained by comparing raw gene expression values using edgeR GLM statistical model⁵⁴. For the PDX model, persister cells were compared to cells from the untreated tumor; for MDA-MB468 cells, cells from cluster R2 (persister cells) were compared to cells from cluster R10 (untreated population). Genes were considered significantly overexpressed if the fold change was higher than 3 and the adjusted p-value less than 0.01. For MDA-MB-468 samples, as the number of cells was high, a subset of 500 cells per cluster was subsampled from each cluster for the differential analysis and downstream steps. Intra-cluster correlation scores were calculated using Pearson's correlation score, with a random subsampling of n=500 cells per cluster.

Single-cell lineage barcode extraction from 10× datasets. To detect the lentiviral inserted barcodes in the 10× sequencing data we used custom R scripts. To avoid running scripts on all reads we: 1. used samtools to extract all unmapped reads from the 10× output bam file, 2. used awk to take reads with either a 3′ or 5′ 20 bp match to the constant flanking region of the barcode allowing one mismatch, 3. retained only reads with both a 10× cell barcode and 10× UMI. We then located the 20 bp match to one of the constant flanking regions allowing one mismatch (but filtered out reads where a mismatch was in the first or last base to ensure the barcode is at the expected position). We then further required a 4 bp exact match on the other side of the barcode, and then extracted the 20 bp viral barcode, read name, 10× cell barcode and 10× UMI.

To assign one viral barcode to each 10× cell barcode, we determined a consensus viral barcode for each UMI. For each position in the barcode we returned the most frequently observed base and the proportion of reads supporting this consensus. Barcodes associated with a 10× CB-UMI pair were filtered if the proportion of reads supporting a position was ≤0.5 at ≥3 positions. Next, one viral barcode for each 10× cell barcode was taken as a consensus across all remaining CB-UMI pairs for each 10× cell barcode. We took the most frequently observed base for each position and the barcode was filtered if the proportion of UMIs supporting a position is ≤0.5 at ≥3 positions. Finally, we checked whether the viral barcodes were in our barcode library and excluded them if not. UMAPs were colored according to lineage identity, for cells each color corresponding to a unique viral barcode. For comparison with bulk datasets, pseudo-bulk barcode frequencies were computed and normalized to 10,000 total barcodes/sample, as for bulk.

Bulk barcode pre-processing. The analysis pipeline was performed as previously published⁵⁵. In brief, using R-3.4.0 (R Development Core Team (2019) http://www.R-project.org), raw reads were first filtered for perfect to the input index-and common-sequences using XCALIBR (https://github.com/NKI-GCF/xcalibr) and filtered against the barcode reference list. Correlation between technical (PCR) replicates (Extended FIG. 3j) was used as quality control: samples were then normalized and filtered for a Spearman correlation between replicates higher than 0.6 and barcodes present in only one of the two replicates were set to zero. The mean of the replicates was used for downstream analysis.

Bulk and single-cell lineage barcode analysis. Barcode frequencies were transformed with asinh. Normalized frequencies from bulk and single-cell datasets were clustered using hierarchical clustering based on Spearman correlation and Ward method. Frequencies across time points and conditions were compared with a Spearman correlation coefficient and associated p-value. To test whether barcode frequencies within DMSO and 5-FU treated cells correspond to a random sampling of the initial untreated population, we used proportionate sampling PPS to simulate an in silico barcode frequency vector from a consensus barcode frequency vector of the initial population—obtained from n=6 drawings—and compared simulated and observed frequencies as above. For single-cell datasets, diversity was defined as the fraction of unique barcodes within the detected barcodes for a given cluster or cell population.

Bulk ChIP-seq analysis and consensus peak annotations. Raw sequencing files were mapped in single-end mode using bowtie with options ‘-k1-m 1’^{56,57} against the human genome (hg38). PCR duplicates were removed using Picard Mark Duplicates function (GitHub Repository. http://broadinstitute.github.io/picard/). In order to define a consensus annotation specific to our MDA-MB-468 model, peaks were first called on each of all bulk MDA-MB-468 samples, both DMSO and 5-FU treated, against their respective inputs using Zerone⁵⁶ with a confidence of 95% and window size of 1,000 bp for H3K27me3 mark and 500 bp for H3K4me3 mark. Peaks were further merged together when closer than 10,000 bp. For H3K4me3 this defined the consensus peak annotation with a total of 29,714 peaks. For H3K27me3, peaks were further filtered to refine annotation: (i) only keeping peaks having signal in at least two samples and (ii) removing small peaks (<2,000bp) with overestimated signal due to window size normalization. A total of 9,568 consensus peaks were found for H3K27me3 landscapes in 5-FU and DMSO treated cells, defining the H3K27me3 consensus peak annotation.

Single-cell ChIP-seq read processing. The single-cell ChIP-seq sequencing files were preprocessed using our single-cell ChIP-seq dedicated pipeline (https://github.com/vallotlab/scChiPseq_DataEngineering).

Each #Read 2 was first splitted into a cell barcode sequence composed of the first 79 nucleotides and the last 22 nucleotides corresponding to genomic DNA. Full #Read 1 and genomic DNA of #Read 2 were mapped in paired-end mode to hg38 whole genome using STAR (v2.6.0c) with parameters ‘—alignEndsType EndToEnd—outFilterMultimapScoreRange 2—win AnchorMultimapNmax 1000—alignIntronMax 1—peOverlapNbasesMin 10—alignMatesGapMax 450’ for PDX model and against hg38 only for MDA-MB-468 cell line, by keeping only reads having no more than one reportable alignments and 2 mismatches. For each barcode (i.e cell), reads with identical #Read 1 starting sites were marked as duplicates, probably emerging from reverse-transcription or PCR duplicates. #Read 1 sequences paired with unmapped #Read 2, and falling within the same 50 bp-window, were further stacked into one read, as possibly originating from PCR duplicates or from the same nucleosome.

For H3K27me3 and H3K4me3 experiments in MDA-MB-468 cells, reads were counted according to two annotations: (i) within consensus peak annotation (used in FIG. 2a-d, 2f, Extended FIG. 5b, e), and (ii) within a TSS-based annotation (used in FIG. 2e,g, 3a-b, Extended FIG. 5 j, 6a-d), comprising 52,138 regions of 10 kbp centered around TSS of all transcripts of protein coding and IncRNAs from Gencode v34⁵⁸. For PDX untreated tumors, H3K27me3 and H3K4me3 experiments, reads were counted according to TSS-based annotation. For all experiments and annotations, only cells with a coverage over 1,000 reads were kept for downstream analysis, see Extended Table 3 for sample and cell numbers.

Single-cell Chip-seq Filtering, Dimensionality Reduction and Clustering. Qc filtering, dimensionality reduction, and clustering were done using ChromSCape⁵⁹, (https://github.com/vallotlab/ChromSCape) with default parameters for H3K4me3 datasets, resulting 1,345 cells with signal over 4,983 TSS. For H3K27me3 datasets, minimum coverage was increased to 3,000 reads/cell and for each sample the number of cells was randomly downsampled at 500 cells per sample to ensure equal contribution of each datasets to dimensionality reduction. The resulting matrix contains 3,576 cells with signal over 8,858 peaks.

In order to exclude from the subsequent analysis known copy number variation (CNV) regions between samples, CNV regions previously identified using ChromHMM⁶⁰ on the input of bulk experiment of MDA-MB-468 samples were used by ChromSCape as regions to exclude from the analysis. Coverage tracks of metacells for scChIP-seq were obtained by aggregating the signal of single-cells into cumulative signals in each cluster. We define a group of cells as being more ‘synchronous’ regarding a set of genes (e.g. persister genes) if they have a significantly higher number of genes with H3K4me3 signal, according to a Wilcoxon non-parametric rank test.

Differential Analysis of H3k27me3 Chromatin Landscapes Genome-wide. These analyses were done using consensus peak annotations, to assess for genome-wide changes without a priori on gene annotation. For each scChIP-seq datasets, for each cluster (E1, E2, E4), pseudo-bulk samples were generated by summing up reads from individual cells provided there were more than 50 cells in the sample in the given cluster. Pseudo-bulk scChIPseq signals are normalized by the total number of reads for all cells of the corresponding population (‘persister’ or ‘untreated’). For each loci, both pseudo-bulk tracks are shown at the same magnification, with the same range for the y-axis to enable comparison between pseudo-bulk tracks.

We performed two differential analysis based on counts within the consensus peak annotation: (i) one to define the specific chromatin changes in persister cells versus untreated cells (FIG. 2c-d, Extended FIG. 5e), where we compared pseudo-bulks and bulks of persister cells to pseudo-bulks and bulks of untreated cells, and (ii) one to compare chromatin landscapes of subpopulations within the untreated populations (Extended FIG. 5e). For (i), as persister cells grouped within one cluster, we combined n=2 pseudo-bulks to n=4 bulk matrices from biological replicates to perform differential analysis using Limma package⁶¹. Peaks with a log2FC over 1 and under −1 and an adjusted p-value below 0.1 were considered significantly enriched or depleted of H3K27me3 in persister cells. For FIG. 2c, we used a generic hg38 genome gene/TSS annotation that classifies regions into categories, e.g. gene TSS, intergenic or enhancer regions. For each category we test whether this category is significantly more prevalent in differentially enriched peaks between persister and untreated states versus in all peaks. The ‘enrichment’ metric is the log2 (number of differential peaks in the category/total number of peaks in that category). Fisher's exact test was used to compare the localization of depleted H3K27me3 peaks in respect to gene annotation.

Epigenomics and transcriptome data comparison. To integrate epigenomic and trancriptome data (SI_Table 4, FIG. 3c), for each gene we combined both TSS-based and peak-based differential analysis (considering peaks closer than 1 kbp to the TSS) with the same thresholds as above. We represented this integration as a donut plot (FIG. 3c), taking into consideration all persister genes n=168 and adding independently, for each, information on H3K27me3 status upon 5-FU treatment (depleted in TSS or nearby peak, or unchanged), on bivalent status in untreated cells (see related section for thresholds) and on presence in the top 100 of CheA3 predicted TFs (see below).

Gene regulatory networks. In order to test whether persister genes are co-regulated by master regulators, we ran CHeA3³³ data mining algorithm (from TF-target interaction based on multiple sources, e.g. ENCODE, GTEx co-expression, ReMap ChIP-seq, EnrichR, ARCHS4 co-expression and ChIP-seq from the literature) to find TFs with regulons enriched in persister genes in vitro and in PDXs. In order to create a background control to assess the quality of the ranking score given by CHeA 3, we ran ChEA 3 for 1000 random gene sets, expressed in our scRNA-seq data and of the same size. The inverse of the ChEA3 score of the top ranking 100 TF regulons enriched in our persister genes were significantly greater than for the random background gene sets for all models (one-sided T-test p. values with respectively for MDA-MB-468, PDX_95, PDX_39 and PDX_172: 2.2e-16, 5.8e-08, 6.9e-3 & 6.9e-05).

Chromatin indexing analysis. The bulk chromatin indexing sequencing files were first demultiplexed by matching the first 8 bases of #Read 2 without any mismatches to the 8-bp long index of each sample from a pool of 5 samples. The same demultiplexing was done for the corresponding inputs. Afterwards, mapping and demultiplexing was done as in bulk ChIP-seq (see above). Relative total amounts of immunoprecipitated DNA were determined as the ratio of the number of reads in the IP by the number of reads in the corresponding input for each sample within the pool. Coverage tracks were normalized with this ratio.

Sequential ChIP-seq analysis. Fastq files for primary (ChIP) and secondary (ChIP-reChIP) immunoprecipitation were processed as for bulk ChIP-seq (see above). For the MDA-MB-468 cell line, sequential ChIP-seq was processed with H3K27me3 as primary ChIP then H3K4me3 as secondary ChIP or vice versa. For other in vitro models and PDXs only the latter H3K4me3->H3K27me3 way was kept as peaks were more easily identifiable in the secondary ChIP profiles. IgG secondary ChIP was used as a negative control.

For the H3K27me3->H3K4me3 reChIP datasets, peaks were called on the secondary ChIP (H3K4me3 or IgG) with MACS262 using the primary H3K27me3 signal as control and with parameters ‘macs 2 callpeak—call-summits −p 0.1—nomodel—extsize 300’. Summits closer to each other by 1,000 bp were merged using Bedtools63. Only peaks overlapping both the TSS annotation and the H3K27me3 consensus peak annotation obtained from ChIP-seq experiments (see above) were kept to focus on TSS chromatin landscapes. A ratio and associated p-value for each peak was calculated as follows:
(i) Reads were counted within the 2 kbp region around the peak summit (“peak”) as well as in the 20 kbp region around peak (“locus”) in each ChIP-reChIP. A ratio of “peak”/“locus” was calculated in order to control for the relative increase in signal in this region (presence of a peak).
(ii) Reads were counted in the 2 kbp region around the summits (“peak”) as well as in the 500 kbp region around peak (“area”) in the primary ChIP and the ChIP-reChIP to create a contingency table.

A Fisher exact test was performed on the contingency table to reject the null hypothesis that the number of reads in “peaks” compared to reads in “area” is greater in the ChIP-reChIP than in the primary ChIP. P-values were adjusted for multiple testing using the Benjamini-Hochberg procedure⁶⁴.

In order to choose adequate thresholds for adjusted p-value and “peak”/“locus”, we calculated the number of false positives for p-values ranging from 0.1 to 0.001 and ratios ranging from 10 to 25% (Extended FIG. 6f), using H3K27me3/IgG ChIP and ChIP-reChIP as a negative control. An adjusted p-value threshold of 0.001 and peak ratio threshold of 15% was chosen to minimize false positives peaks and resulted in 1,266 bivalent peaks covering 1,547 TSS.

For the H3K4me3->H3K27me3 reChIP datasets, peaks were first called on primary ChIP using MACS2 without control with parameters ‘—call-summits −p 0.01—nomodel—extsize 300’. The number of reads in the region 2.5 kbp upstream and downstream of each peak were counted in the primary and secondary ChIP. Reads were normalized by total library size. First, the ratio between secondary and primary ChIP were calculated for each peak and then the odd-ratio between each TSS and it's 60 closest neighbours were calculated from the ratios. In order for a TSS to be considered bivalent, the odd ratio of a given peak compared to the 60 closest neighbour peaks must be greater than 4. Then, A Fisher exact test was performed on the contingency table to reject the null hypothesis that the number of reads in secondary compared to reads in primary ChIP is greater in the given peak than in it's 60 neighbor peaks. P-values were adjusted for multiple testing using the Benjamini-Hochberg procedure⁶⁴. In order to choose adequate thresholds for adjusted p-value and “peak”/“locus”, we calculated the number of false positives for p-values ranging from 0.001 to 1e-30, using H3K4me3/IgG ChIP and ChIP-reChIP as a negative control. Adjusted p-value thresholds were always lower than 0.001 and were defined so that we obtained the greatest number of bivalent peaks in the H3K4me3/H3K27me3 experiment compared to the H3K4me3/IgG negative control.

The comparative coverage tracks were generated by calculating the log2 ratio of secondary ChIP versus primary ChIP using Deeptools bamCompare and then smoothed. For each loci, H3K27me3/H3K4me3 and H3K27me3/IgG or H3K4me3/H3K27me3 and H3K4me3/IgG tracks are shown at the same magnification and with the same range for the y-axis for comparison between tracks.

Bulk CUT&TAG of patient tumors to assess bivalency. Fastq files for H3K27me3 and H3K4me3 were processed as for bulk ChIP-seq (see above). For each sample, reads were counted on the TSS annotation and normalized using log2 RPKM. Then, only the top 15% most covered TSS were kept for H3K4me3 and H3K27me3. Bivalent TSS were taken as the intersection of these highly covered TSS.

Gene set analysis. For all gene set analysis, we applied hypergeometric tests to identify gene sets enriched within significantly overexpressed genes (scRNA), genes devoid of H3K27me3 (scChIP-seq) or bivalent genes (Sequential ChIP-seq, bulk Cut&Tag) from MSigDB v5 database⁶⁵, correcting for multiple testing with the Benjamini-Hochberg procedure. Gene sets with an adjusted p-value below 0.1 were considered significantly enriched. The gene background universe for hypergeometric testing was the entire set of expressed genes for scRNA or the 32,937 genes present in Gencode for scChIP-seq or bivalent gene lists. The tests were performed on all lists, but we display only the relevant following lists: ‘c2_curated’ related to breast (searching for ‘MAMMARY’ or ‘BREAST’), ‘c2_curated’ related to ‘KEGG’, ‘c5_GO’ and ‘c7_hallmark’, filtering out genetic-event related lists (containing ‘AMPLICON’).

When displaying gene set analysis of multiple samples, we first selected gene sets significantly enriched in 3/3 of PDXs or in vitro samples or at least 7/9 samples for human tumors. Then, gene sets were ranked by the average adjusted p-values, and only the top 5 gene sets of each category were displayed. The dotplots representing pathway enrichment across multiple samples were done using clusteRprofileR⁶⁶. The gene ratio stands for the fraction of genes belonging to each pathway. For the PDX and in vitro experiments, the −log10 adjusted p-value of the main models are displayed, respectively PDX_95 and MDA-MB-468. In order to calculate the significativity of overlaps when we compared multiple set of pathways, we used the Exact Test of Multi-set intersections⁶⁷.

Whole Exome Sequencing data analysis. The WES sequencing files were mapped using bwa-mem⁶⁸ to the human genome (hg19). Reads falling in the targeted regions were then filtered based on their mapping quality and PCR duplicates were removed. Local Indel Realignment and Base Score Recalibration was applied to deduplicated reads using GATK⁶⁹. Somatic variants were called with Mutect2⁷⁰ using early passage 16 (p16) and a series of patient blood samples as reference (referred as “blood”, see Extended FIG. 4a). At this step, only mutations labeled as ‘PASS’ or ‘t_lod_fstar’ were kept and additional filters were applied based on http://best-practices-for-processing-hts-data.readthedocs.io/en/latest/mutect2_pitfalls.html. Using p16 as the “normal” sample allowed to directly obtain somatic variants acquired after treatment or not to 5-FU, but we hypothesized that some germline variants might be wrongly called somatic variants so we filtered out variants that were reported in ExAC Non Finnish European database, effectively removing 231 variants possibly germline. For MDA-MB-468 persisters & resistant samples, mutations also present in one of the untreated samples were removed. Mutations occurring in a breast cancer driver gene list from¹³ was used to annotate mutations falling in driver genes.

GAP⁷¹ was used to calculate with precision absolute copy number and B allele frequencies (BAF) taking depth of coverage and allele frequency from a set of known germline variants from the⁷² as inputs, and using “blood” as normal sample. Palimpsest⁷³ was then used to calculate the Cancer Cell Fraction (CCF) of each mutation in each sample, i.e. the proportion of cells in the population bearing the mutation, correcting by purity, BAF and absolute copy number of the segment. Then mutations were classified in either ‘subclonal’ or ‘clonal’ depending on their CCF. Finally, de novo mutational signatures were obtained from the mutations context and matched to a set of known signatures from COSMIC v2 (https://cancer.sanger.ac.uk/cosmic/signatures_v2) that were observed in breast cancer (i.e. signatures 1, 2, 3, 8, 13, 17, 18, 20, 26 & 30).

### Results

We focused on mechanisms of drug tolerance in residual TNBC, for which patients have the poorest outcome. Resistance to adjuvant chemotherapy cannot be easily studied as biopsies are not routinely performed when the disease progresses (FIG. 1a). To circumvent these limitations, we modeled, in vivo and in vitro, phenotypes of drug-response observed in patients. In vivo, we first treated three patient-derived xenograft (PDX) models—PDX_95, PDX_39 & PDX_172—established from patients with residual TNBC^{16,18}, with Capecitabine, the standard of care for residual breast tumors (FIG. 1b, Extended FIG. 1). After the first round of chemotherapy treatment, mice displayed a pathological complete response (pCR), but tumors eventually recurred (‘recurrent’) and mice were treated again with chemotherapy, to which tumors responded to various extents, some maintaining constant tumor volume under treatment (‘resistant’) (FIG. 1b). These recurrent tumors potentially arose from persister cells, surviving initial chemotherapy treatment⁸. We isolated patient-derived persister cells by pooling the fat pad from mice with pCR (from 4 to 14, Extended FIGS. 1a, 1f & 1l). To phenocopy a clinical situation of partial response, we also generated ‘residual’ tumors for one model (PDX_95, n=2) (Extended FIG. 1a) by administering half the dose of Capecitabine.

In vitro, we treated an initially chemosensitive TNBC cell line (MDA-MB-468), with Fluorouracil (5-FU)¹⁹, the active metabolite of Capecitabine, which is not converted in vitro. We drove independently three pools of cells to chemoresistance with prolonged 5-FU treatment (>15 weeks, FIG. 1e). After 3 weeks, only few cells survived drug insult (0.01% of the initial population, Extended FIG. 2 a) and started proliferating again under chemotherapy after 10-15 days (FIG. 1e). Over 15 weeks, populations of resistant cells emerged, with an IC50 to 5-FU over 4-fold higher than untreated population and doubling times comparable to untreated cells (Extended FIG. 2b).

To characterize transcriptomic evolution of untreated cells towards chemotolerance and subsequently chemoresistance, we performed single-cell RNA-seq (scRNA-seq) in both cell lines and PDX models (FIG. 1c, 1f, Extended FIGS. 1 & 2). In vivo, scRNA-seq was mandatory to identify the rare human persister cells among the vast majority of stromal mouse cells. Out of the fat pad, we typically isolated hundreds of persister cells per mouse. In vivo, persister cells from different mice grouped within one or two expression clusters (Extended FIGS. 1b, 1g-h & 1m-n). In vitro, diverse cell populations (res #1, 2 and 3) with distinct expression programs, originated from the pool of persister cells, all grouped within common clusters across experiments (R2/R4 Extended FIG. 2c). In vivo and in vitro, persister cells recurrently activated a set of common pathways compared to untreated cells (FIG. 1c-f, Extended FIG. 1c-d, 1i-j, 1o-p Extended FIG. 2d-e). Originating from KRT5-expressing cancer cells, persister cells recurrently activated sets of genes further establishing basal cell identity, such as KRT14 (FIGS. 1c, 1f, Extended 1j & 1p). Compared to untreated cells, persister cells in vivo and in vitro also showed an activation of genes associated with the Epithelial-to-Mesenchymal Transition (EMT, FIG. 1c-f, Extended FIGS. 1c-d, 1j & 1p, Extended FIGS. 2d& 2f)—such as TAGLN, an actin-binding protein, previously shown to promote metastasis through EMT²⁰, and NNMT, characteristic of the metabolic changes that accompany EMT²¹⁻²³. Persister cells also activated genes involved in the TNFalpha/NF-KB pathway. Part of the persister expression program remained patient-specific: in PDX_95 and in vitro persister cells showed for example an activation of the TGF-β pathway with the expression of multiple players including ligands and receptors (FIGS. 1c & 1f, SI Tables 1-4).

In vivo, we showed that persister and residual tumor cells actually clustered together (FIG. 1c, cluster R4), thereby sharing common expression features, suggesting similar mechanisms of chemotolerance independent of the residual burden. Yet we detected a higher number of cells in G0/G1 within persister populations than in residual or untreated tumors (Extended FIG. 1e-k-q), a characteristic recapitulated in vitro (Extended FIG. 2g) and in line with previous reports^{1,4,11,24}. In vitro, we identified two clusters of persister cells (clusters R2 and R4), that differ by their expression of additional EMT markers such as CDH2 (FIG. 1f) and TWIST1. Early individual persisters (day 33) solely belonged to cluster R2/CDH2— whereas growing persisters could either belong to R2/CDH2- or R4/CDH2+ (FIG. 1f and Extended FIG. 2c). Overall, we identified both in vivo and in vitro a reservoir of persister basal cells with EMT markers and activated NF-KB pathway. NF-KB signaling pathway and EMT associated-genes were proposed as potential drivers of chemoresistance in various tumor types, including lung^{25,26}, pancreatic²⁷, breast^{28,29}. Here we pinpoint NF-KB and EMT pathway activation as the earliest common molecular events at the onset of chemotolerance in TNBC, defining a common Achilles' heel to target chemotolerant cells before they phenotypically diversify.

To follow clonal evolution under therapeutic stress, we had initially introduced unique genetic barcodes in untreated MDA-MB-468 cells prior to our experiments (Extended FIG. 3a). We leveraged our previous barcoding method³⁰ to allow robust detection of barcodes within scRNA-seq data (FIG. 1g), as shown by the number of cells with a detected lineage barcode (Extended FIG. 3b). In addition, we verified that barcodes frequencies detected in scRNA-seq data recapitulated those detected in bulk, confirming the sensitivity of barcode detection in scRNA-seq data (Extended FIG. 3c). By combining detection of lineage barcode and expression programs at single-cell resolution, we were able to monitor clonal evolution over the course of the treatment and within each expression clusters (FIG. 1g). If non-cycling persisters (day 33) were multi-clonal (62% of unique barcodes), barcode diversity rapidly decreased overtime within the CDH2−/R2 persister cluster, and R6 and R8 chemoresistant clusters were constituted of few clones (3 and 4 respectively). Barcode diversity within the CDH2−/R2 cluster was significantly higher than in CDH2+/R4 cluster across all experiments and time points (average of 41% versus 8% unique barcode, p=1.6e-2), demonstrating that if the drug-tolerant state is multi-clonal, only rare persister cells switch to the CDH2+ state.

To test if the lineages that persist were selected within the untreated population, we next compared barcode frequencies between the starting population and the 5-FU or DMSO-treated cells using additional bulk experiments (Experiment #3, Extended FIG. 3d-e). If surviving cells had no particular predisposition then they should resemble a random draw of the initial untreated population (day 0, Extended FIG. 3f). In contrast to DMSO-treated cells, barcode frequencies of the 5-FU treated cell deviated from this random scenario (Extended FIG. 3g, r=0.68 and r=0.29 respectively), indicating that a fraction of lineages present in the untreated population have a predisposition to tolerate chemotherapy. Using our single-cell barcoding dataset, we were able to identify within the untreated populations, future persisters (n=143, for which the lineage barcode is found both in persisters and untreated cells) and compare them to ‘non-persisting’ cells (n=201, Extended FIG. 3h-i). The only transcriptomic differences between those two cell populations were the over-expression of S100A2, a calcium binding protein, and LDHB, a lactate dehydrogenase, in future persister cells, prior to treatment. These results suggest that a difference in the metabolism of cells could be an indicator of their potential for persistence.

To hamper the chemo-driven clonal evolution of cancer cells, we next investigated the molecular basis of such rapid phenotypic evolution. Using whole-exome sequencing (Extended FIG. 4a), we first analyzed mutations, copy-number alterations (CNA) and related mutational signatures acquired by persister and resistant cell populations since the onset of 5-FU treatment. We could not identify any recurrent mutations across experiments (Extended FIG. 4b), or any CNA (amplifications or homozygous deletions) or recurrent mutations affecting known driver genes of breast cancer in any population¹³. Only a minor fraction of mutations found in persister cells were attributed to 5-FU exposure (mutational signature 17³¹) in contrast to resistant cell populations where over 50% of acquired mutations are associated to 5-FU (p<10⁻¹⁰, Extended FIG. 4c). These results indicated that chemo-related mutations are acquired over a timeframe that is not compatible with the rapid phenotypic evolution seen in persister cells. Finally, computing cancer cell fractions for each mutation, we confirmed that persister populations are extensively multi-clonal (Extended FIG. 4d), in line with the lineage barcoding results.

We next investigated changes in epigenomes during chemotherapy treatment. Using single-cell profiling (scChIP-seq), we observed that H3K27me3 epigenomes faithfully captured the evolution of cell states with chemotherapy (FIG. 2a, Extended FIG. 5a and SI Table 4). Persister cells shared a common H3K27me3 epigenome (cluster E1, FIG. 2b, Extended FIG. 5b), in contrast to resistant cells split in clusters E1 and E3. In comparison to untreated cells, cells from cluster E1 showed recurrent redistribution of H3K27me3 methylation, the highest changes (|log2FC|>2 and adjusted p-value<10⁻¹) occurring specifically at transcription start sites (TSS) and gene bodies (FIG. 2c) and corresponding to a loss of H3K27me3 enrichment (75 regions with log2FC<-2, and 2 regions with log2FC>2). This depletion was associated with the highest changes in gene expression observed by scRNA-seq (FIG. 2d and Extended FIG. 5c) and to the transcriptional de-repression of 22% of persister genes—defined as genes overexpressed in persister versus untreated cells—such as TGFB1, FOXQ1, a transcription factor driving EMT (FIG. 2e, SI Table 4). These epigenomic changes were not necessarily kept in resistant cell populations (Extended FIG. 5d), suggesting the existence of transient epigenomic features in persister cells.

In the untreated cells, two epigenomic subclones (E2 & E4 clusters, FIG. 2b) were recurrently identified indicative of an epigenomic heterogeneity in this population (n=3 experiments). In contrast to cells from cluster E4 (median correlation score r=−0.34), a fraction of cells within cluster E2 shared epigenomic similarities with cells from E1 (FIG. 2f, 49/381 cells over r=0.20, p<2.2e-16). Yet these similarities did not affect persister genes (Extended FIG. 5e), and cells remained discernible from the pool of persister cells (no cells from E2 with r score over median r in E1, p<2.2e-16). This suggests that cells from E2 could fuel the persister population when exposed to chemotherapy, with the need of chemo-induced chromatin changes to achieve tolerance. In addition, we also detected rare cells with a persister epigenomic signature, but in only one of our three experiments (60/976 cells—Extended FIG. 5b), suggesting that spontaneous transition to H3K27me3 drug-tolerant state rarely occurs in the absence of chemotherapy.

To test whether H3K27me3 enrichment was the lock to the persister expression program in untreated cells, we treated cancer cells with the EZH2 inhibitor (EZH2i-1) UNC1999³², to deplete H3K27me3 from cells, in the absence of chemotherapy. EZH2i-1 treatment phenocopied drug-tolerant state to chemotherapy as expression fold-changes induced by EZH2i-1 were specifically correlated to those induced by chemotherapy exposure at early time points in persister cells (FIG. 2g, Extended FIG. 5f, r=0.71 versus r=0.31 with changes in resistant cells). Furthermore, we observed that EZH2i-1 was sufficient to lead to the activation of 62% of persister genes with depletion of H3 K27me3 upon 5-FU treatment (23/37 genes), suggesting that H3K27me3 was the sole lock to their activation (FIG. 2h and Extended FIG. 5g). EZH2i-1 was also sufficient to lead to the over-expression of 60% of persister genes independently of any H3 K27me3 enrichment in untreated cells (78/131 genes), such as KRT14, suggesting that these genes might be targets of H3K27me3-regulated persister genes. To further test this hypothesis, we explored the existence of potential ‘master’ transcription factors (TFs) within the persister genes. Using CheA3³³, we identified three transcription factors, FOXQ1, FOSL1 and N 2RF2, that respectively target 34%, 42% and 29% of the persister genes (Extended FIG. 5 h-i). All 3 are H3K27me3-regulated persister genes, with a loss of H3K27me3 upon 5-FU (Extended FIG. 5j) and re-expressed by EZH2i-1 treatment, proposing H3K27me3-regulated TF as potential drivers of the persister expression program.

As we observed H3K27me3 changes upon 5-FU treatment precisely at TSS, we further explored the evolution of chromatin modifications at TSS, focusing on H3K4me3, a permissive histone mark shown to accumulate over TSS with active transcription. In contrast to single-cell H3K27me3 epigenomes which were sufficient to separate cell states along treatment (FIG. 2a), individual H3K4me3 epigenomes of untreated and persister cells were indiscernible (FIG. 3a-b). Sparse H3K4me3 enrichment was already observed in untreated cells at the TSS of persister genes (p<10⁻¹⁵, compared to a set of non-expressed genes, Extended FIG. 6a-b). In individual persister cells, H3K4me3 enrichment was significantly more synchronous at the TSS of persister genes with more persister genes simultaneously marked by H3K4me3 in the same cell than in untreated cells (p=4.0e-2, Extended FIG. 6c-d). Based on these results, we reasoned that in untreated cells H3K4me3 and H3K27me3 could either accumulate on the same TSS but in different cells, or H3K4me3 could accumulate together with H3K27me3 in a subset of cells on the same TSS.

To test whether H3K4me3 could co-exist with H3K27me3 in the same individual cells prior to chemotherapy exposure, we performed successive immunoprecipitation of H3K27me3 with H3K4me3 (or vice-versa) or H3K27me3 (or H3K4me3) with isotype control (IgG) on mono-nucleosome chromatin. In MDA-MB-468 cells, we detected n=1,547 transcription start sites significantly enriched in DNA immunoprecipitated with both H3K27me3 and H3K4me3, compared to the control (H3K27me3/IgG) precipitated fraction (peak-ratio>0.15, q-value<10⁻³, Extended FIG. 6e-h). We found that bivalent chromatin in untreated cells was detected at TSS of genes associated to mammary stem cell and EMT pathways, as well as various developmental pathways (e.g Hedgehog pathway, Extended FIG. 6h-i). The majority of H3K27me3-regulated persister genes (28 out of 37), including the 3 candidate master TFs, were found in a bivalent chromatin configuration in the untreated cell population (e. g TGF-β1, FOXQ1, FOSL1, FIG. 3b-c, Extended FIG. 6d and SI Table 4). We next studied bivalent chromatin landscapes in additional TNBC cell lines (HCC38 & BT20, Extended FIG. 6i), in our 3 PDX models and n=9 patient samples-3 of which correspond to the tumors used for PDX derivation (Patient_95, Patient_39 & Patient_172) (FIG. 3d-f, Extended FIG. 7a-h). We confirm the existence of bivalent programs in all patient tumors, and show that paired PDX models faithfully recapitulate bivalency of patient samples (Extended FIG. 7a-f). Bivalent chromatin is also found at genes implicated in EMT and mammary stem cell identity, and developmental pathways (FIG. 3f, Extended FIG. 7h) and at candidate master TFs of the patient-derived persister programs—FOXQ1, KLF4 or TFCP2L1—(FIG. 3d-e, Extended FIG. 7g). With a continuum of epigenomic datasets, from patients to PDX, we demonstrate that cancer cells display a set of bivalent TSS, which could lead to the rapid activation of an entire persister expression program upon therapeutic stress.

To further test if H3K27me3 was a lock to the emergence of persister cells under chemotherapy exposure, we next depleted H3K27me3 from epigenomes prior to chemotherapy treatment in three TNBC cell lines (MDA-MB-468, BT20 and HCC38, Extended FIG. 8). Using EZH2i-1, in addition to an inactive isomer (UNC240034) and a second EZH2i (GSK126³⁵ referred to as EZH2i−2), we showed that erasing H3K27me3—without perturbing EZH2 protein levels—increased the number of persister cells with both EZH2 inhibitors, while the inactive isomer had no effect (FIG. 4a, Extended FIG. 8). By comparing bulk lineage barcode frequencies across conditions, we further demonstrated that EZH2 inhibitors were actually affecting cell fate under chemotherapy exposure, by rescuing the biased lineage frequency induced by 5-FU treatment (FIG. 4b, Extended FIG. 8c). We observed that co-treatment with EZH2i-1 and EZH2i-2 increased correlation scores between lineage frequencies in 5-FU and DMSO treated population, whereas co-treatment with the inactive isomer had no effect on lineage frequencies (FIG. 4b, Extended FIG. 8c). Performing combined single-cell transcriptomics and lineage tracing as in FIG. 1, we then compared barcode diversity across expression clusters under 5-FU exposure with or without EZH2i-1, termed “EZH2i-1 persister” and “persister” respectively (FIG. 4c-e). We observed that co-treating cells with EZH2i-1 significantly increased the diversity in lineage barcodes in CDH2+ persister populations, identified in FIG. 1 (R3 cluster, p<10⁻⁴ and over 2 fold-increase, FIG. 4e). Altogether, our results showed that H3K27me3 depletion with EZH2 inhibitors rescued the biased lineage frequency observed under chemotherapy treatment, and enabled a wider variety of cells to switch to the CDH2+ drug-tolerant state. Overall, depleting H3K27me3 from untreated cells not only launched a persister-like expression program, but it also enhanced the potential of each cancer cell to tolerate chemotherapy.

Finally, we tested our ability to inhibit the emergence of persister cells by preventing the depletion of H3K27me3 under chemotherapy exposure using a KDM 6A/B—“Lysine demethylase 6A/B” inhibitor (KDM6A/Bi—GSK-J4³⁶) simultaneously to chemotherapy. We tested in vitro and in vivo the ability of GSK-J4 to reduce the pool of persister cells upon chemotherapy exposure, reasoning that a reduced number of persister cells would increase the delay to recurrence. In vitro, in opposition to EZH2i, co-treatment with KDM6A/Bi led to a decrease in the number of persisters at day 21 and further completely abolished the growth of persister cells under 5-FU at day 60, whereas it had no effect on untreated cancer cells (FIG. 5a, Extended FIG. 7b-c). Interestingly, administrating KDM6i once persister cells have emerged, rather than in combination to chemotherapy, is inefficient (FIG. 5b, Extended FIG. 7d), demonstrating that the switch from untreated to drug-tolerant state, rather than the drug-tolerant state itself, was sensitive to KDM6i. These results were confirmed in two additional TNBC cell lines, BT20 and HCC38 (Extended FIG. 7e-f). In vivo, our objective was to test the potential of KDM6i to limit the emergence of persister cells, when administered simultaneously to chemotherapy. We compared the disease-free survival time of mice treated with Capecitabine alone (n=25) or in combination with KDM6i (n=25). The administration of KDM6i did not inhibit tumor progression in absence of chemotherapy nor was toxic for the mice (FIG. 5c). However administered in combination to chemotherapy, it significantly increased the delay to recurrence (FIG. 5d, p=4.0e-2) in comparison to chemotherapy alone. Our in vitro and in vivo results together suggest that a fraction of cancer cells could need to actively demethylate H3K27me3 residues to tolerate chemotherapy. These results are consistent with a mechanism where persister genes would be repressed by H3K27me3 in untreated cells, and primed with stochastic H3K4me3 in a subset of cells, with the loss of H3K27me3 unlocking the transition to tolerance.

In conclusion, our study shows that H3K27me3 landscapes are determinants of cell fate upon chemotherapy exposure in TNBC. We demonstrate that, prior to treatment, cells display bivalent chromatin landscapes priming the persister expression program with H3K4me3 and H3K27me3. In other words, genes are ready to be activated with H3K4me3, but are repressed with H3K27me3 that remains the lock to the activation of the persister expression program. Using EZH2 inhibitors and lineage tracing strategies, we further demonstrate that, depleting H3K27me3 from the genome rescues the cell fate bias normally observed upon chemotherapy insult; cells have an equal probability of surviving initial chemotherapy insult. Persister cells could be cells without H3K27me3 or the one releasing the H3K27me3 lock, or a mixture of both phenomena as shown here: co-treating cells with a H3K27me3 demethylase inhibitor together with 5-FU, we reduced, but not totally abrogated the number of persister cells. We propose that combining chemotherapy with histone demethylase inhibitors at the onset of chemotherapy exposure will delay recurrence by decreasing the pool of persister cells. Several studies had started to interrogate which epigenetic modifiers could regulate expression programs of persister or resistant cells^{11,35,37,38}. Here, we show that even prior to treatment, the epigenome is already a key player, with a priming of the persister program. Our findings highlight how chromatin landscapes can shape the potential of cancer cells for chemotolerance.

Our results that depletion of H3K27me3 with EZH2 inhibitors—at the onset of treatment—enhances chemotolerance leaves open questions regarding the role of H3K27me3 landscapes in cancer evolution, and the usage of EZH2i in cancer treatment. Depending on the context and the timing of administration, EZH2i might have drastically different effects, and such subtleties should be carefully considered before therapy combination. In contrast to our observations at the onset of treatment, TNBC cancer cells with long-term acquired chemo-resistance have been shown to display DNA hypomethylation and large H3K27me3 over transposable elements, hence a vulnerability to EZH2i³⁹. EZH2i were also recently shown to lead to MHC Class I upregulation in cancer cells, thereby showing beneficial immunotherapeutic effects^{40,41}. If such cell plasticity represents a therapeutic opportunity, our results also show that EZH2i could also lead, in some contexts, to the activation of a set of genes driving drug-persistence.

Isolated examples of bivalent promoters had been found in tumor cells^{42,43}, here we exhaustively map bivalent promoters genome-wide, revealing epigenomic priming of mammary stem cell genes and signaling pathways of known resistance pathways in TNBC⁴⁴, including Hedgehog, WNT, TGF-β, ATP-binding cassette drug transporters pathways. Such epigenomic priming is reminiscent of developmental bivalency priming mechanisms⁴⁵ found in stem cells prior to differentiation and appears key for the rapid activation of the genes upon therapeutic stress. Remains to be understood, how only a minority of bivalent genes are targeted by gene reactivation upon chemotherapy exposure—which could be associated to the nature of the treatment itself—and whether such priming mechanisms could be shared across cancer types. Determining the precise addressing mechanisms that target H3K27me3 and H3K4me3 writers and readers to TSS of cancer-type bivalent genes under therapeutic stress, will be instrumental in the future to identify dedicated co-factors which could serve as therapeutic targets to further limit the emergence of persister cells.

The present invention has been described in terms of specific embodiments, which are illustrative of the invention and not to be construed as limiting. More generally, it will be appreciated by persons skilled in the art that the present invention is not limited by what has been particularly shown and/or described hereinabove.

Reference numerals in the claims do not limit their protective scope.

Use of the verbs “to comprise”, “to include”, “to be composed of”, or any other variant, as well as their respective conjugations, does not exclude the presence of elements other than those stated.

Use of the article “a”, “an” or “the” preceding an element does not exclude the presence of a plurality of such elements.

### BIBLIOGRAPHIC DATA

1. Vallette, F. M. et al. Dormant, quiescent, tolerant and persister cells: Four synonyms for the same target in cancer. Biochemical Pharmacology 162, 169-176 (2019).
2. Shen, S., Vagner, S. & Robert, C. Persistent Cancer Cells: The Deadly Survivors. Cell 183, 860-874 (2020).
3. Li, X. et al. Triple-negative breast cancer has worse overall survival and cause-specific survival than non-triple-negative breast cancer. Breast Cancer Res. Treat. 161, 279-287 (2017).
4. Sharma, S. V. et al. A Chromatin-Mediated Reversible Drug-Tolerant State in Cancer Cell Subpopulations. Cell 141, 69-80 (2010).
5. Echeverria, G. V. et al. Resistance to neoadjuvant chemotherapy in triple-negative breast cancer mediated by a reversible drug-tolerant state. Sci. Transl. Med. 11, eaav0936 (2019).

6. Ramirez, M. et al. Diverse drug-resistance mechanisms can emerge from drug-tolerant cancer persister cells. Nat Commun 7, 10690 (2016).
7. Shaffer, S. M. et al. Rare cell variability and drug-induced reprogramming as a mode of cancer drug resistance. Nature 546, 431-435 (2017).
8. Cortazar, P. et al. Pathological complete response and long-term clinical benefit in breast cancer: the CTNeoBC pooled analysis. The Lancet 384, 164-172 (2014).
9. Hata, A. N. et al. Tumor cells can follow distinct evolutionary paths to become resistant to epidermal growth factor receptor inhibition. Nat Med 22, 262-269 (2016).
10. Kim, C. et al. Chemoresistance Evolution in Triple-Negative Breast Cancer Delineated by Single-Cell Sequencing. Cell 173, 879-893.e13 (2018).
11. Liau, B. B. et al. Adaptive Chromatin Remodeling Drives Glioblastoma Stem Cell Plasticity and Drug Tolerance. Cell Stem Cell 20, 233-246.e7 (2017).
12. Rambow, F. et al. Toward Minimal Residual Disease-Directed Therapy in Melanoma. Cell 174, 843-855.e19 (2018).
13. Nik-Zainal, S. et al. Landscape of somatic mutations in 560 breast cancer whole-genome sequences. Nature 534, 47-54 (2016).
14. Mazor, T. et al. DNA Methylation and Somatic Mutations Converge on the Cell Cycle and Define Similar Evolutionary Histories in Brain Tumors. Cancer Cell 28, 307-317 (2015).
15. Gaiti, F. et al. Epigenetic evolution and lineage histories of chronic lymphocytic leukaemia. Nature 569, 576-580 (2019).
16. Grosselin, K. et al. High-throughput single-cell ChIP-seq identifies heterogeneity of chromatin states in breast cancer. Nat. Genet. 51, 1060-1066 (2019).
17. Kaya-Okur, H. S. et al. CUT&Tag for efficient epigenomic profiling of small samples and single cells. Nat Commun 10, 1930 (2019).
18. Marangoni, E. et al. A New Model of Patient Tumor-Derived Breast Cancer Xenografts for Preclinical Assays. Clinical Cancer Research 13, 3989-3998 (2007)
19. Longley, D. B., Harkin, D. P. & Johnston, P. G. 5-fluorouracil: mechanisms of action and clinical strategies. Nat. Rev. Cancer 3, 330-338 (2003).
20. Chen, Z. et al. TGF-β-induced transgelin promotes bladder cancer metastasis by regulating epithelial-mesenchymal transition and invadopodia formation. EBioMedicine 47, 208-220 (2019).
21. Ulanovskaya, O. A., Zuhl, A. M. & Cravatt, B. F. NNMT promotes epigenetic remodeling in cancer by creating a metabolic methylation sink. Nat. Chem. Biol. 9, 300-306 (2013).
22. Shaul, Y. D. et al. Dihydropyrimidine Accumulation Is Required for the Epithelial-Mesenchymal Transition. Cell 158, 1094-1109 (2014).
23. Liang, L., Zeng, M., Pan, H., Liu, H. & He, Y. Nicotinamide N-methyltransferase promotes epithelial-mesenchymal transition in gastric cancer cells by activating transforming growth factor-β1 expression. Oncol Lett (2018) doi: 10.3892/ol.2018.7885.
24. Oren, Y. et al. Cycling cancer persister cells arise from lineages with distinct transcriptional and metabolic programs. http://biorxiv. org/lookup/doi/10.1101/2020.06.05.136358 (2020) doi: 10.1101/2020.06.05.136358.
25. Fischer, K. R. et al. Epithelial-to-mesenchymal transition is not required for lung metastasis but contributes to chemoresistance. Nature 527, 472-476 (2015).
26. Ryan, S.-L. et al. Targeting NF-KB-mediated inflammatory pathways in cisplatin-resistant NSCLC. Lung Cancer 135, 217-227 (2019).
27. Zheng, X. et al. Epithelial-to-mesenchymal transition is dispensable for metastasis but induces chemoresistance in pancreatic cancer. Nature 527, 525-530 (2015).
28. Zeng, D. et al. Inhibition of Notch 1 reverses EMT and chemoresistance to cisplatin via direct downregulation of MCAM in triple-negative breast cancer cells. Int. J. Cancer 147, 490-504 (2020).
29. Godwin, P. et al. Targeting Nuclear Factor-Kappa B to Overcome Resistance to Chemotherapy. Front. Oncol. 3, (2013).
30. Eisele, A. S. et al. Erythropoietin directly affects single hematopoietic stem cell differentiation after transplantation. http://biorxiv. org/lookup/doi/10.1101/2020.04.20.050146 (2020) doi: 10.1101/2020.04.20.050146.
31 Christensen, S. et al. 5-Fluorouracil treatment induces characteristic T>G mutations in human cancer. Nat Commun 10, 4571 (2019).
32. Konze, K. D. et al. An Orally Bioavailable Chemical Probe of the Lysine Methyltransferases EZH2 and EZH1. ACS Chem. Biol. 8, 1324-1334 (2013).
33. Keenan, A. B. et al. ChEA 3: transcription factor enrichment analysis by orthogonal omics integration. Nucleic Acids Research 47, W212-W224 (2019).
34. Chagraoui, H. et al. SCL/TAL1 cooperates with Polycomb RYBP-PRC1 to suppress alternative lineages in blood-fated cells. Nat Commun 9, 5375 (2018).
35. Göllner, S. et al. Loss of the histone methyltransferase EZH2 induces resistance to multiple drugs in acute myeloid leukemia. Nat Med 23, 69-78 (2017).
36 Kruidenier, L. et al. A selective jumonji H3K 27 demethylase inhibitor modulates the proinflammatory macrophage response. Nature 488, 404-408 (2012).
37. Vinogradova, M. et al. An inhibitor of KDM 5 demethylases reduces survival of drug-tolerant cancer cells. Nat Chem Biol 12, 531-538 (2016).
38. Hinohara, K. et al. KDM5 Histone Demethylase Activity Links Cellular Transcriptomic Heterogeneity to Therapeutic Resistance. Cancer Cell 34, 939-953.e9 (2018).
39. Deblois, G. et al. Epigenetic Switch-Induced Viral Mimicry Evasion in Chemotherapy-Resistant Breast Cancer. Cancer Discov 10, 1312-1329 (2020).
40. Burr, M. L. et al. An Evolutionarily Conserved Function of Polycomb Silences the MHC Class I Antigen Presentation Pathway and Enables Immune Evasion in Cancer. Cancer Cell 36, 385-401.e8 (2019).
41. Mahadevan, N. R. et al. Intrinsic immunogenicity of small cell lung carcinoma revealed by its cellular plasticity. Cancer Discov candisc.0913.2020 (2021) doi: 10.1158/2159-8290.CD-20-0913.
42. Hahn, M. A. et al. Loss of the Polycomb Mark from Bivalent Promoters Leads to Activation of Cancer-Promoting Genes in Colorectal Tumors. Cancer Research 74, 3617-3629 (2014).
43. Chaffer, C. L. et al. Poised Chromatin at the ZEB 1 Promoter Enables Breast Cancer Cell Plasticity and Enhances Tumorigenicity. Cell 154, 61-74 (2013).
44. Nedeljković, M. & Damjanović, A. Mechanisms of Chemotherapy Resistance in Triple-Negative Breast Cancer—How We Can Rise to the Challenge. Cells 8, 957 (2019).
45. Bernstein, B. E. et al. A Bivalent Chromatin Structure Marks Key Developmental Genes in Embryonic Stem Cells. Cell 125, 315-326 (2006).

1. A method for determining a risk of cancer recurrence in a human subject who had or has a cancer, wherein the method comprises the steps of:
determining in cancer cell, optionally tumor cells exposed to a chemotherapeutic agent, issued from a biological sample previously obtained from the subject whether at least one genetic biomarker selected from the group comprising or consisting of LDHB, S100A2, KRT14, TAGLN, NNMT, FOXQ1, FOSL1, NR2F2, KLF4 and TFCP2L1 is over-expressed as compared to a reference level,
identifying the presence of persister cell in the biological sample as cancer cell over-expressing at least one genetic biomarker, optionally all genetic biomarkers, which expression has been measured,
classifying the subject as being at risk to have cancer recurrence when persister cell has been identified in the biological sample.

2. The method according to claim 1, wherein the persister cell over expresses:
at least LDHB and S100A2; and/or
at least KRT14, TAGLN, and NNMT; and/or
at least FOSL1 and KLF4, optionally at least FOXQ1, FOSL1, NR2F2 and KLF4, FOXQ1, FOSL1, NR2F2, KLF4 and TFCP2L1.

3. The method according to claim 1 wherein the expression of at least LDHB and S110A2 is determined, optionally wherein the expression of all genetic biomarkers is determined.

4. The method according to claim 3, wherein persister cells over-express all genetic biomarkers from the list consisting of S100A2, LDHB, KRT14, TAGLN, NNMT, FOXQ1, FOSL1, NR2F2, KLF4 and TFCP2L1.

5. The method according to claim 1, further comprising the quantification of H3K27me3 within the cancer cell, optionally the quantification of H3K27me3 associated with the promoter of at least one gene encoding a genetic biomarker selected from the group comprising or consisting of LDHB, S100A2, KRT14, TAGLN, NNMT, FOXQ1, FOSL1, NR2F2, KLF4 and TFCP2L1.

6. A method for determining a risk of cancer recurrence in a human subject who had or has a cancer, wherein the method comprises the steps of:
determining in cancer cell, optionally tumor cells exposed to a chemotherapeutic agent, issued from a biological sample previously obtained from the subject the presence or the absence of persister cells, wherein a persister cell has a quantity of H3K27me3 lower than a reference level,
classifying the subject as being at risk to have cancer recurrence when persister cells have been identified in the biological sample.

7. The method according to claim 1, wherein the subject had or has a cancer selected from the list consisting of bladder cancer, bone cancer, brain cancer, breast cancer, cervical cancer, colon cancer, esophageal cancer, gastric cancer, head & neck cancers, hodgkin's lymphoma, leukemia, liver cancer, lung cancer, melanoma, mesothelioma, multiple myeloma, myelodysplastic syndrome, non-hodgkin's lymphoma, ovarian cancer, pancreatic cancer, prostate cancer, rectal cancer, renal cancer, sarcoma, skin cancer, testicular cancer, thyroid cancer or uterine cancer, optionally a breast cancer, or a Triple-Negative Breast Cancer (TNBC).

8. A method for measuring the presence or absence of persister cells within a biological sample comprising cancer cells, optionally tumor cells exposed to a chemotherapeutic agent, issued from a biological sample previously obtained from at least one patient who has or had a cancer, wherein the method comprises:
determining in cancer cell whether at least one genetic biomarker selected from the group comprising or consisting of S100A2, LDHB, KRT14, TAGLN, NNMT, FOXQ1, FOSL1, NR2F2, KLF4 and TFCP2L1 is over-expressed as compared to a reference level,
identifying the presence of persister cell in the biological sample as cancer cell over-expressing at least one genetic biomarker, optionally all genetic biomarkers, which expression has been measured, or the absence of persister cells in the biological sample as no cancer cell over-expressing the genetic biomarker(s) which expression has been measured.

9. A method for treating a human subject who had or has a cancer, said method comprising:
determining in cancer cell, optionally tumor cells exposed to a chemotherapeutic agent, issued from a biological sample previously obtained from the subject whether at least one genetic biomarker selected from the group comprising or consisting of S100A2, LDHB, KRT14, TAGLN, NNMT, FOXQ1, FOSL1, NR2F2, KLF4 and TFCP2L1 is over-expressed as compared to a reference level,
identifying the presence of persister cell in the biological sample as cancer cell over-expressing the one or more genetic biomarkers, optionally all genetic biomarkers, which expression has been measured,
when persister cell has been identified in the biological sample, administering to the subject an inhibitor of lysine demethylase or histone demethylase, optionally an inhibitor of lysine demethylase 6, of lysine demethylase 6 A/B or of H3K27me3 demethylase; and/or administering to the subject an inhibitor of S100A2, LDHB, KRT14, TAGLN, NNMT, FOXQ1, FOSL1, NR2F2, KLF4 or TFCP2L1.

10. The method according to claim 9, wherein persister cell expresses at least:
S100A2 and LDHB; and/or
at least KRT14, TAGLN, and NNMT; and/or
at least FOSL1 and KLF4, optionally at least FOXQ1, FOSL1, NR2F2 and KLF4, FOXQ1, FOSL1, NR2F2, KLF4 and TFCP2L1,
S100A2, LDHB, KRT14, TAGLN, NNMT, FOXQ1, FOSL1, NR2F2, KLF4 and TFCP2L1.

11. A method for treating or preventing cancer recurrence in a human subject who had or has a cancer, said method comprising:
determining in cancer cell, optionally tumor cells exposed to a chemotherapeutic agent, issued from a biological sample previously obtained from the subject whether at least one genetic biomarker selected from the group comprising or consisting of S100A2, LDHB, KRT14, TAGLN, NNMT, FOXQ1, FOSL1, NR2F2, KLF4 and TFCP2L1 is over-expressed as compared to a reference level,
identifying the presence of persister cell in the biological sample as cancer cell over-expressing the one or more genetic biomarkers, optionally all genetic biomarkers, which expression has been measured,
when persister cells have been identified within the biological sample, administering to the subject an inhibitor of lysine demethylase or histone demethylase, optionally an inhibitor of lysine demethylase 6, of lysine demethylase 6 A/B or of H3K27me3 demethylase and/or administering to the subject an inhibitor of S100A2, LDHB, KRT14, TAGLN, NNMT, FOXQ1, FOSL1, NR2F2, KLF4 or TFCP2L1.

12. The method according to claim 9, wherein the subject has or had a cancer selected from the group consisting of bladder cancer, bone cancer, brain cancer, breast cancer, cervical cancer, colon cancer, esophageal cancer, gastric cancer, head & neck cancers, hodgkin's lymphoma, leukemia, liver cancer, lung cancer, melanoma, mesothelioma, multiple myeloma, myelodysplastic syndrome, non-hodgkin's lymphoma, ovarian cancer, pancreatic cancer, prostate cancer, rectal cancer, renal cancer, sarcoma, skin cancer, testicular cancer, thyroid cancer or uterine cancer, optionally a breast cancer, and or a Triple-Negative Breast Cancer (TNBC).

13. A method for preventing or reducing cancer recurrence by targeting persister cells with an effective amount of a chemotherapeutic agent and an inhibitor of lysine demethylase, optionally an inhibitor of lysine demethylase 6, more of lysine demethylase 6 A/B or of H3K27me3 demethylase.

14. A method for reducing the number of persister cells upon chemotherapy exposure comprising administering to a patient in need thereof an effective amount of an inhibitor of lysine demethylase, optionally an inhibitor of lysine demethylase 6, of lysine demethylase 6 A/B or of H3K27me3 demethylase.

15. The method according to claim 9, wherein the inhibitor is administered in combination with a chemotherapeutic agent, optionally simultaneously, wherein the chemotherapeutic agent a thymidylate synthase (TS) inhibitor,

16. A kit for performing the method according to claim 1.

17. The kit according to claim 16, which comprises reagents for measuring the expression of at least one genetic biomarker selected from the group comprising or consisting of LDHB, S100A2, KRT14, TAGLN, NNMT, FOXQ1, FOSL1, NR2F2, KLF4 and TFCP2L1 in a sample previously obtained from a human subject who had or has a cancer.
